(19) United States
(12) Patent Application Publication
(10) Pub. No.: US 2013/0172342 A1
(43) Pub. Date: Jul. 4, 2013

(54) BENZIMIDAZOLE DERIVATIVES AS SELECTIVE BLOCKERS OF PERSISTENT SODIUM CURRENT

(71) Applicant: Allergan, Inc., Irvine, CA (US)

(72) Inventors: George R. Ehring, Huntington Beach, CA (US); Michael E. Garst, Newport Beach, CA (US); Alfred A. Avey, JR., Eugene, OR (US); Lloyd Jay Dolby, Eugene, OR (US); Shervin Esfandiari, Eugene, OR (US); Vivian R. MacKenzie, Eugene, OR (US); Jeremiah Andrew Marsden, Eugene, OR (US); David C. Muchmore, Eugene, OR (US)

(73) Assignee: Allergan, Inc., Irvine, CA (US)

(21) Appl. No.: 13/727,945

(22) Filed: Dec. 27, 2012

Related U.S. Application Data
(60) Provisional application No. 61/580,980, filed on Dec. 28, 2011.

(51) Int. Cl. C07D 235/14

(52) U.S. Cl. CPC C07D 235/14
USPC 514/234.5; 544/139; 546/199; 514/322; 544/370; 514/254.06

(57) ABSTRACT

The present invention is directed to methods of treating diseases or conditions mediated by elevated persistent sodium channel, such as ocular disorders, pain, multiple sclerosis, and seizure disorders utilizing a compound of Formula I

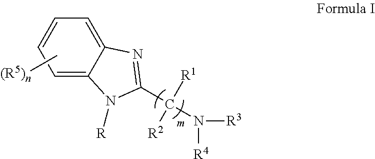

or a pharmaceutically acceptable salt thereof or a pharmaceutical composition comprising said compound, wherein variables R, R¹, R², R³, R⁴, R⁵, m, and n in Formula I are as defined herein.

### CROSS REFERENCE TO RELATED APPLICATIONS

This application claims the benefit of U.S. Provisional Application Ser. No. 61/580,980 filed Dec. 28, 2011, which is incorporated by reference herein in its entirety.

### FIELD OF THE INVENTION

The present invention is directed to benzimidazole derivative compounds, pharmaceutical compositions comprising the compounds and methods of using the compounds and the pharmaceutical compositions that selectively reduce persistent sodium current in a mammal and in treating diseases or conditions that involve elevated persistent sodium current in a mammal, such as chronic pain, epileptic seizure, and ocular/retinal diseases, as well as other diseases and conditions associated with elevated persistent sodium current.

### BACKGROUND OF THE INVENTION

Voltage-gated sodium channels, transmembrane proteins that initiate action potentials in nerve, muscle and other electrically excitable cells, are a necessary component of normal sensation, emotions, thoughts and movements (Catterall, W. A., Nature (2001), Vol. 409, pp. 988-990). These channels consist of a highly processed alpha subunit that is associated with auxiliary beta subunits. The pore-forming alpha subunit is sufficient for channel function, but the kinetics and voltage dependence of channel gating are in part modified by the beta subunits (Goldin et al., Neuron (2000), Vol. 28, pp. 365-368). Each alpha-subunit contains four homologous domains, I to IV, each with six predicted transmembrane segments. The alpha-subunit of the sodium channel, forming the ion-conducting pore and containing the voltage sensors regulating sodium ion conduction has a relative molecular mass of 260,000. Electrophysiological recording, biochemical purification, and molecular cloning have identified nine different sodium channel alpha subunits and four beta subunits (Yu, F. H., et al., Sci. STKE (2004), 253; and Yu, F. H., et al., Neurosci. (2003), 20:7577-85).

The hallmarks of sodium channels include rapid activation and inactivation when the voltage across the plasma membrane of an excitable cell is depolarized (voltage-dependent gating), and efficient and selective conduction of sodium ions through conducting pores intrinsic to the structure of the protein (Sato, C., et al., Nature (2001), 409:1047-1051). At negative or hyperpolarized membrane potentials, sodium channels are closed. Following membrane depolarization, sodium channels open rapidly and then inactivate. Channels only conduct currents in the open state and, once inactivated, have to return to the resting state, favored by membrane hyperpolarization, before they can reopen. Different sodium channel subtypes vary in the voltage range over which they activate and inactivate as well as their activation and inactivation kinetics. Alterations in the gating mechanism of sodium channels can contribute to disease. A loss of the ability to inactivate results in a sustained influx of sodium into cells. This process is termed a persistent sodium current. Depending on the amplitude of this current changes in cell excitability or triggered cell death can occur.

The sodium channel family of proteins has been extensively studied and shown to be involved in a number of vital body functions. Research in this area has identified variants of the alpha subunits that result in major changes in channel function and activities, which can ultimately lead to major pathophysiological conditions. Implicit with function, this family of proteins is considered a prime point of therapeutic intervention. Naᵥ1.1 and Naᵥ1.2 are highly expressed in the brain (Raymond, C. K., et al., J. Biol. Chem. (2004), 279(44):46234-41) and retina, and are vital to normal brain function. In humans, mutations in Naᵥ1.1 and Naᵥ1.2 result in severe epileptic states and in some cases mental decline (Rhodes, T. H., et al., Proc. Natl. Acad. Sci. USA (2004), 101(30):11147-52; Kamiya, K., et al., J. Biol. Chem. (2004), 24(11):2690-8; Pereira, S., et al., Neurology (2004), 63(1):191-2). As such both channels have been considered as validated targets for the treatment of epilepsy (see PCT Published Patent Publication No. WO 01/38564).

Voltage-gated sodium channels comprise a family of proteins designated from Naᵥ1.3 through Naᵥ1.9. The sodium channel isoforms show differential expression throughout the central and peripheral nervous system. (Catterall, W. A., Nature (2001), Vol. 409, pp. 988-990; Goldin et al., Neuron (2000), Vol. 28, pp. 365-368; Ehring, George R., et al. “Diversity of Expression of Voltage-Gated Sodium Channels in the Rat Retina.” ARVO Meeting Abstracts 53.6 (2012): 5337; O'Brien, B. J., et al. “Tetrodotoxin-resistant voltage-gated sodium channels Na(v)1.8 and Na(v)1.9 are expressed in the retina.” J Comp Neurol 508.6 (2008): 940-51).

U.S. Pat. No. 7,309,716 (assigned to Vertex Pharmaceuticals) discloses benzimidazole compounds that are useful as inhibitors of voltage-gated sodium channels.

U.S. Pat. Nos. 7,125,908, 7,763,651, 7,767,718, and 8,153,645 (all assigned to Allergan) disclose methods of treating chronic pain in a mammal by administering to the mammal an effective amount of a selective persistent sodium channel antagonist that has at least 20-fold selectivity for persistent sodium current relative to transient sodium current.

U.S. Pat. No. 7,754,440 (assigned to Allergan) discloses a method for identifying a selective persistent Na⁺ channel blocker by measuring the ability of the blocker to reduce or inhibit a persistent Na⁺ current to a greater degree than a transient Na⁺ current. Aspects of the present method provide Na⁺ depletion/repletion methods for identifying a selective blocker of a persistent Na⁺ channel, hyperpolarization methods for identifying a blocker of a persistent Na⁺ channel, and Na/K ATPase pump inhibitor methods for identifying a selective blocker of a persistent Na⁺ channel.

### SUMMARY OF THE INVENTION

The present invention is directed to a compound selected from the group consisting of:

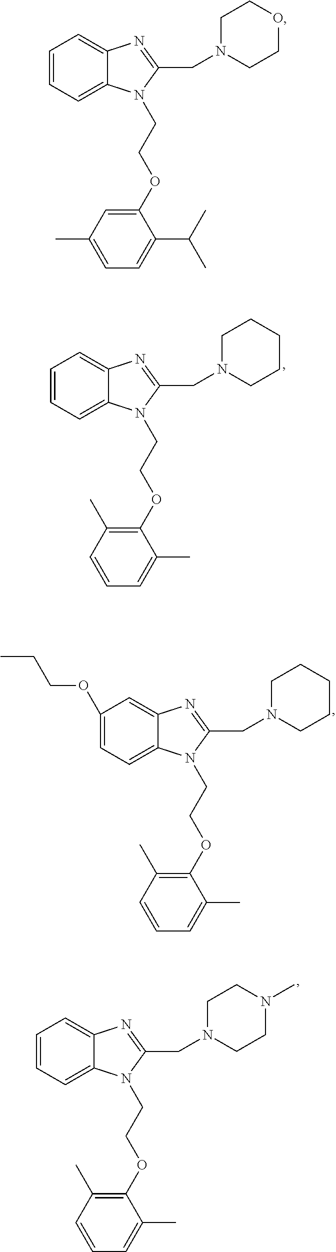 [SMILES: Cc1ccc(C(C)C)c(OCCn2c(CN3CCOCC3)nc3ccccc32)c1 ; Cc1cccc(C)c1OCCn1c(CN2CCCCC2)nc2ccccc21 ; CCCOc1ccc2c(c1)nc(CN1CCCCC1)n2CCOc1c(C)cccc1C ; Cc1cccc(C)c1OCCn1c(CN2CCN(C)CC2)nc2ccccc21]

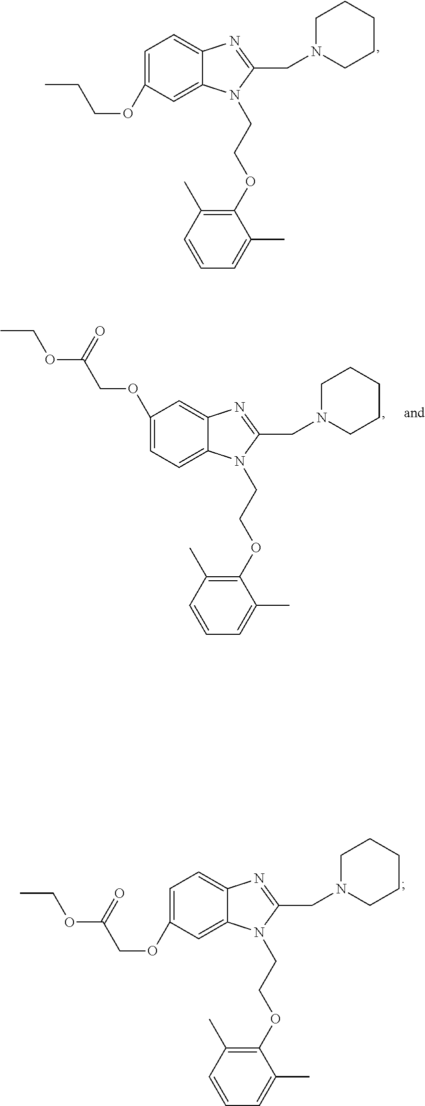 [SMILES: CCCOc1ccc2nc(CN3CCCCC3)n(CCOc3c(C)cccc3C)c2c1 ; CCOC(=O)COc1ccc2c(c1)nc(CN1CCCCC1)n2CCOc1c(C)cccc1C ; CCOC(=O)COc1ccc2nc(CN3CCCCC3)n(CCOc3c(C)cccc3C)c2c1]

or a pharmaceutically acceptable salt thereof.

The present invention is also directed to a pharmaceutical composition comprising at least one of the aforementioned compounds as set forth above, or a pharmaceutically acceptable salt thereof and a pharmaceutically acceptable carrier.

The present invention is also directed to a method of blocking persistent sodium current without affecting transient current in a mammal using a therapeutically effective amount of the compound of Formula I

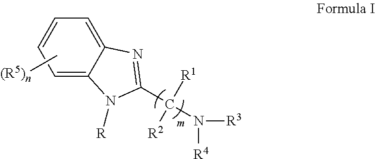

or a pharmaceutically acceptable salt thereof; wherein:

R is C₁₋₆ alkyl, which is unsubstituted or substituted with a substituent selected from the group consisting of —N(C₁₋₆ alkyl)₂, C₆₋₁₂ aryl, C₆₋₁₂ aryloxy, heteroaryl, and —C(═O)—N(R⁶)—C₆₋₁₂ aryl;

each R¹ and R² are independently H or C₁₋₆ alkyl;

R³ and R⁴ together with the nitrogen atoms to which they are shown attached form a five- or six-membered heterocyclyl group selected from the group consisting of pyrrolidinyl, piperidinyl, piperazinyl, and morpholinyl, each of which is independently unsubstituted or substituted with a ring system substituent;

R⁵ is selected from the group consisting of C₁₋₆ alkyl, and C₁₋₆ alkoxy, wherein said C₁₋₆ alkyl and the “alkyl” portion of said C₁₋₆ alkoxy are independently unsubsubstituted or substituted with a substituent selected from the group consisting of hydroxyl, —C(═O)OH, —C(═O)O—C₁₋₆ alkyl, —C(═O)-heterocyclyl, and —N(C₁₋₆ alkyl)₂;

R⁶ is H or C₁₋₆ alkyl;

m is 1 or 2; and

n is 0 or 1.

The present invention is also directed to methods of using the compounds of Formula I and pharmaceutical compositions comprising the compounds of Formula I as set forth above for the treatment of diseases and conditions that are mediated by elevated persistent sodium current, such as pain (especially chronic pain) ocular and retinal disorders, seizure disorders and multiple sclerosis.

### BRIEF DESCRIPTION OF THE DRAWINGS

FIG. 1 shows that Compound #1 (part A) and Compound A (part B) are effective in blocking persistent sodium currents at concentrations that did not affect transient currents. Naᵥ1.3 or Naᵥ1.6 channels were expressed in HEK cells, currents were measured using standard MPC electrophysiological techniques. Data are summarized from at least 3 experiments for each concentration tested and were normalized to the control currents before compound addition. Data are presented as the mean and standard error of the mean (n=3) of the percentage of the control current observed at steady state after compound addition.

### DETAILED DESCRIPTION OF THE INVENTION

### Definitions

As used above, and throughout this disclosure, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, aryl, cycloalkyl, cyano, hydroxy, alkoxy, alkylthio, amino, oxime (e.g., ═N—OH), —NH(alkyl),

—NH(cycloalkyl), —N(alkyl)₂, —O—C(O)-alkyl, —O—C(O)-aryl, —O—C(O)-cycloalkyl, —SF₅, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —SF₅, —OSF₅ (for aryl), —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), —NY₁Y₂, -alkyl-NY₁Y₂, —C(O)NY₁Y₂, —SO₂NY₁Y₂ and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

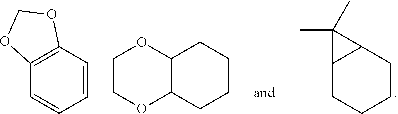

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. Example of such moiety is pyrrolidone:

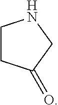

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. Example of such moiety is pyrrolidinone:

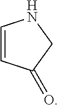

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

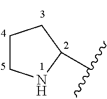 [SMILES: *C1CCCN1]

there is no —OH attached directly to carbons marked 2 and 5.

It should also be noted that tautomeric forms such as, for example, the moieties:

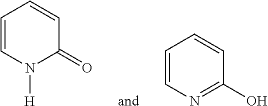 [SMILES: O=c1cccc[nH]1 ; "and" ; Oc1ccccn1]

are considered equivalent in certain embodiments of this invention.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound’ or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like) in sufficient purity to be characterizable by standard analytical techniques described herein or well known to the skilled artisan.

The present invention further includes the compound of formula I in all its isolated forms. Thus, for example, the compound of Formula I is intended to encompass all forms of the compound such as, for example, any solvates, hydrates, stereoisomers, tautomers etc.

The present invention further includes the compound of formula I in its purified form.

It should also be noted that any carbon as well as heteroatom with unsatisfied valences in the text, schemes, examples and Tables herein is assumed to have the sufficient number of hydrogen atom(s) to satisfy the valences. And any one or more of these hydrogen atoms can be deuterium.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American

Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C1-C2)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-(C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula I, and salts, solvates, esters and prodrugs thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T½>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula I, and of the salts, solvates, esters and prodrugs of the compounds of Formula I, are intended to be included in the present invention.

### Embodiments of the Invention

As set forth in the Summary of the Invention, in addition to providing the specific individual compounds as set forth in the Summary, the present invention is also directed to a method of blocking sodium channel in a mammal using a therapeutically effective amount of the compound of Formula I or a pharmaceutically acceptable salt thereof.

In one embodiment, in Formula I, R is C₁₋₆ alkyl, which is unsubstituted or substituted with a substituent selected from the group consisting of C₆₋₁₂ aryloxy and —C(═O)—N(R⁶)—C₆₋₁₂ aryl.

In another embodiment, in Formula I, R is C₁₋₆ alkyl, which is unsubstituted or substituted with a substituent selected from the group consisting of C₆₋₁₂ aryloxy and —C(═O)—N(R⁶)—C₆₋₁₂ aryl; wherein the “aryl” portion of said C₆₋₁₂ aryloxy and —C(═O)—N(R⁶)—C₆₋₁₂ aryl groups is independent unsubstituted or substituted with 1-3 substituents independently selected from the group consisting of halo, C₁₋₆ alkyl, C₂₋₆ alkenyl, and C₁₋₆ alkoxy, wherein the C₁₋₆ alkyl or the “alkyl” portion of said C₁₋₆ alkoxy is independently unsubstituted or substituted with an alkenyl substituent.

In another embodiment, in Formula I, R is C₁₋₆ alkyl which is substituted with a C₆₋₁₂ aryloxy substitutent, and wherein the “aryl” portion of said C₆₋₁₂ aryloxy substitutent is unsubstituted or substituted with 1-2 substituents independently selected from the group consisting of chlro, methyl, isopropyl, methoxy, 1-propenyl, and 2-propenyl.

In another embodiment, in Formula I, R is selected from the group consisting of:

### Ethyl,

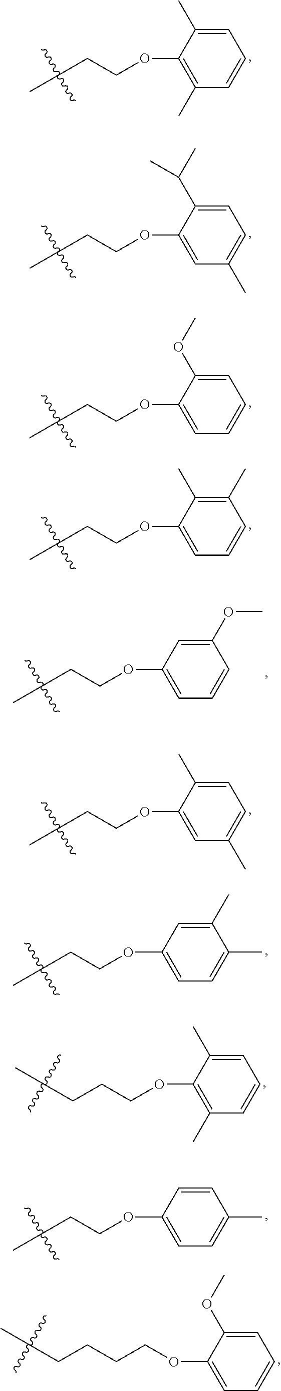

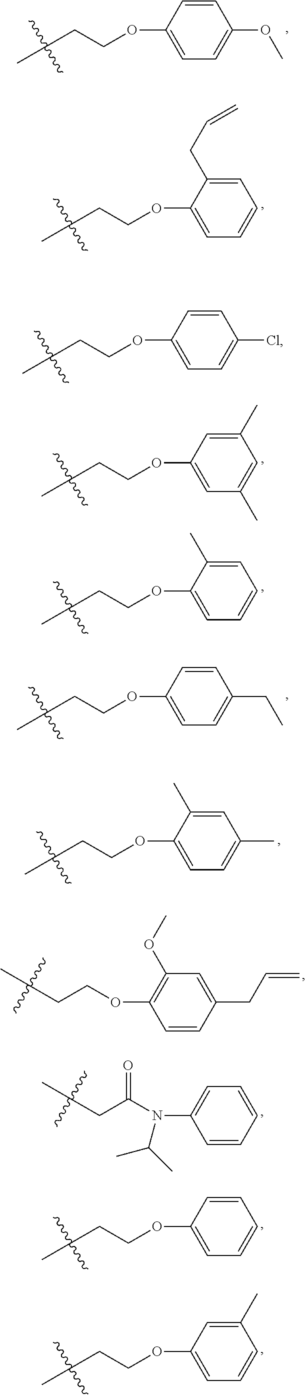

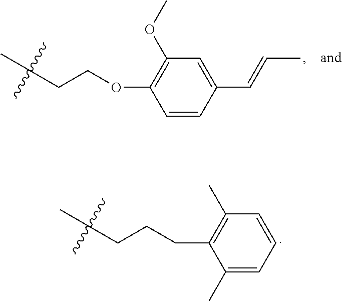 [SMILES: *CCOc1ccc(/C=C/C)cc1OC ; *CCCc1c(C)cccc1C]

In another embodiment, in Formula I, R¹ and R² are both H.

In another embodiment, in Formula I, one of R¹ and R² is C₁₋₆ alkyl, and the other is H.

In another embodiment, in Formula I, one of R¹ and R² is methyl, and the other is H.

In another embodiment, in Formula I, m is 1.

In another embodiment, in Formula I, R³ and R⁴ together with the nitrogen atoms to which they are shown attached form a six-membered heterocyclyl group selected from the group consisting of piperidinyl, and morpholinyl, each of which is independently unsubstituted or substituted with a ring system substituent.

In another embodiment, in Formula I, R³ and R⁴ together with the nitrogen atoms to which they are shown attached form a six-membered heterocyclyl group selected from the group consisting of piperidinyl, and morpholinyl, each of which is independently unsubstituted or substituted with a C₁₋₆ alkyl.

In another embodiment, in Formula I, n is 0.

In another embodiment, in Formula I, n is 1.

In another embodiment, in Formula I, R⁵ is C₁₋₆ alkoxy, wherein the “alkyl” portion of said C₁₋₆ alkoxy is unsubsubstituted or substituted with a —C(═O)O—C₁₋₆ alkyl.

In another embodiment, in Formula I, R⁵ is selected from the group consisting of —O—CH₂CH₂CH₃ and —O—CH₂—C(═O)—O—CH₂CH₃.

In another embodiment, the compound of Formula I is selected from the group consisting of:

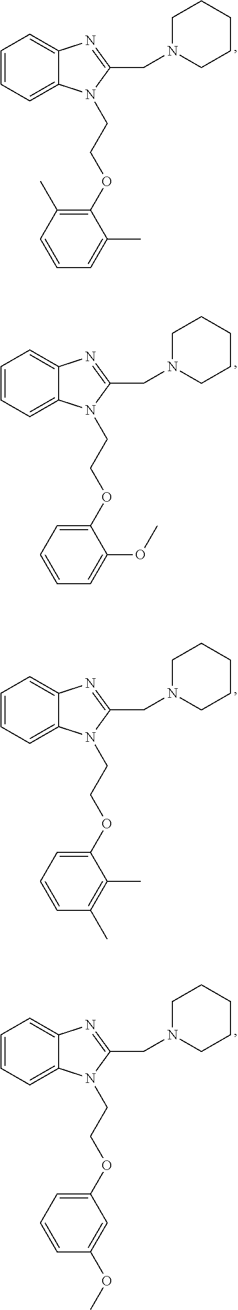 [SMILES: Cc1cccc(C)c1OCCn1c(CN2CCCCC2)nc2ccccc21 ; COc1ccccc1OCCn1c(CN2CCCCC2)nc2ccccc21 ; Cc1cccc(OCCn2c(CN3CCCCC3)nc3ccccc32)c1C ; COc1cccc(OCCn2c(CN3CCCCC3)nc3ccccc32)c1]

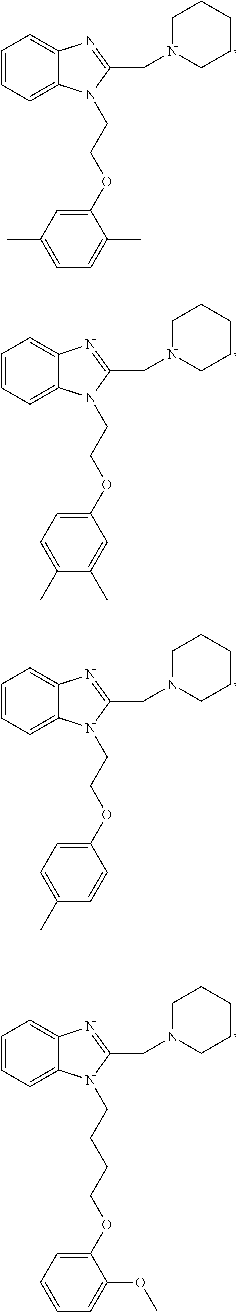 [SMILES: Cc1ccc(C)c(OCCn2c(CN3CCCCC3)nc3ccccc32)c1 ; Cc1ccc(OCCn2c(CN3CCCCC3)nc3ccccc32)cc1C ; Cc1ccc(OCCn2c(CN3CCCCC3)nc3ccccc32)cc1 ; COc1ccccc1OCCCCn1c(CN2CCCCC2)nc2ccccc21]

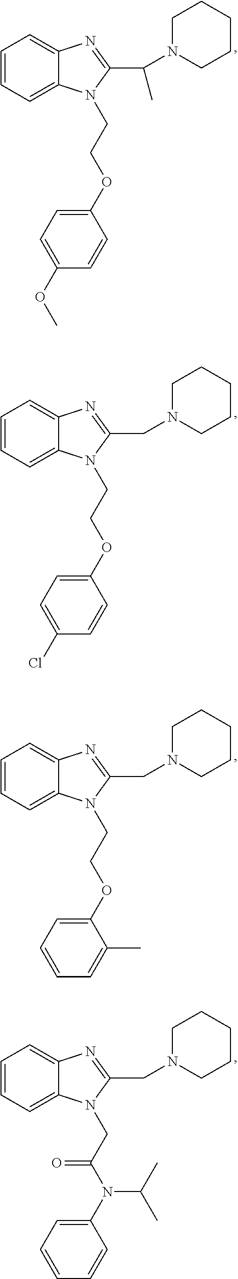 [SMILES: COc1ccc(OCCn2c(C(C)N3CCCCC3)nc3ccccc32)cc1 ; Clc1ccc(OCCn2c(CN3CCCCC3)nc3ccccc32)cc1 ; Cc1ccccc1OCCn1c(CN2CCCCC2)nc2ccccc21 ; CC(C)N(C(=O)Cn1c(CN2CCCCC2)nc2ccccc21)c1ccccc1]

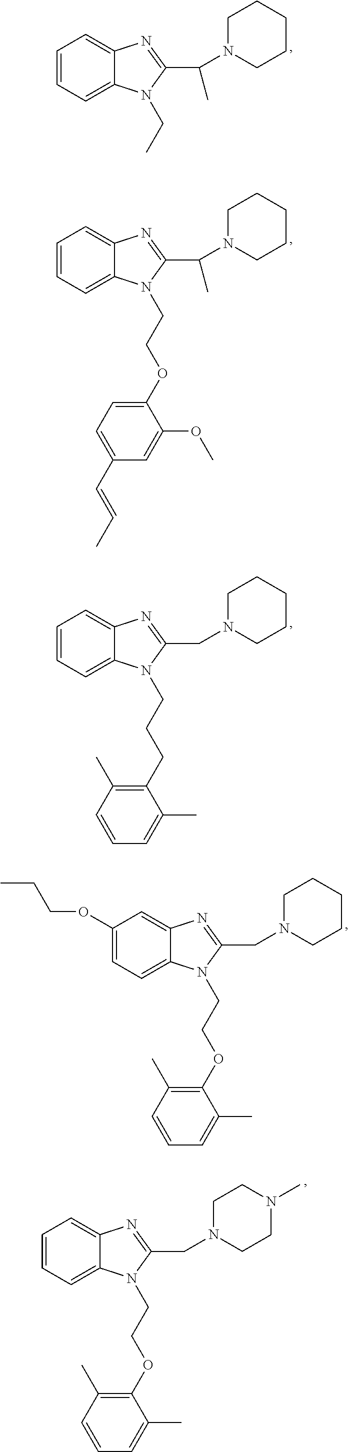 [SMILES: CCn1c(C(C)N2CCCCC2)nc2ccccc21 ; C/C=C/c1ccc(OCCn2c(C(C)N3CCCCC3)nc3ccccc32)c(OC)c1 ; Cc1cccc(C)c1CCCn1c(CN2CCCCC2)nc2ccccc21 ; CCCOc1ccc2c(c1)nc(CN1CCCCC1)n2CCOc1c(C)cccc1C ; Cc1cccc(C)c1OCCn1c(CN2CCN(C)CC2)nc2ccccc21]

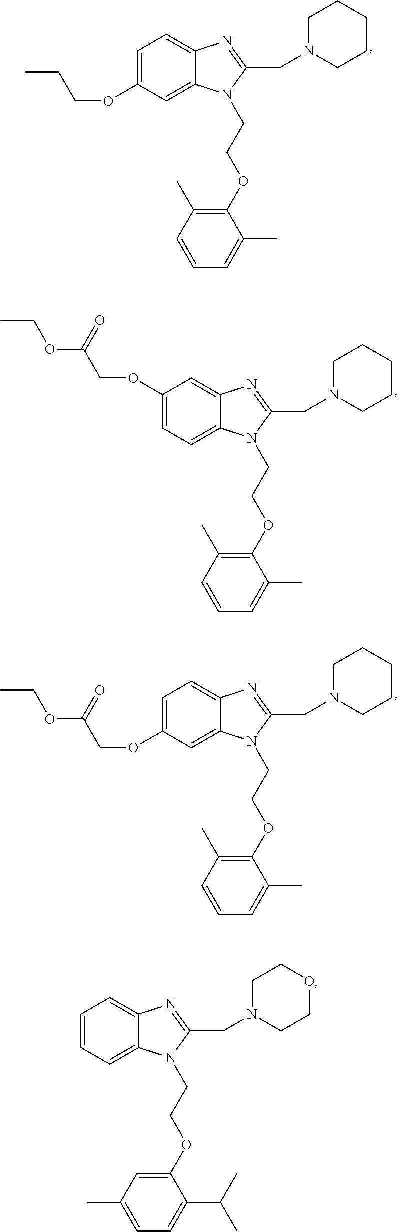 [SMILES: CCCOc1ccc2nc(CN3CCCCC3)n(CCOc3c(C)cccc3C)c2c1 ; CCOC(=O)COc1ccc2c(c1)nc(CN1CCCCC1)n2CCOc1c(C)cccc1C ; CCOC(=O)COc1ccc2nc(CN3CCCCC3)n(CCOc3c(C)cccc3C)c2c1 ; Cc1ccc(C(C)C)c(OCCn2c(CN3CCOCC3)nc3ccccc32)c1]

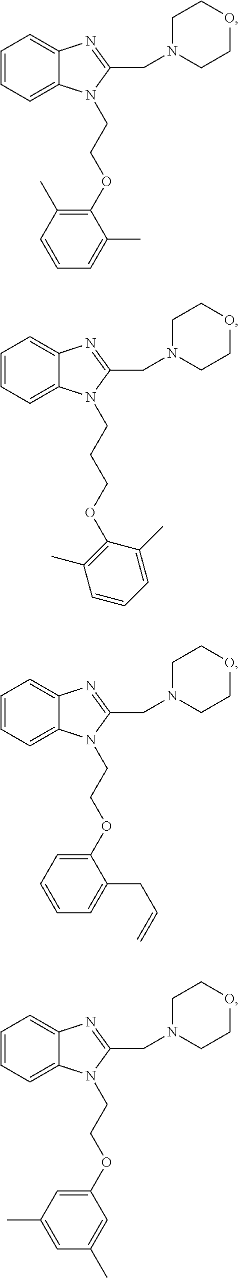 [SMILES: Cc1cccc(C)c1OCCn1c(CN2CCOCC2)nc2ccccc21 ; Cc1cccc(C)c1OCCCn1c(CN2CCOCC2)nc2ccccc21 ; C=CCc1ccccc1OCCn1c(CN2CCOCC2)nc2ccccc21 ; Cc1cc(C)cc(OCCn2c(CN3CCOCC3)nc3ccccc32)c1]

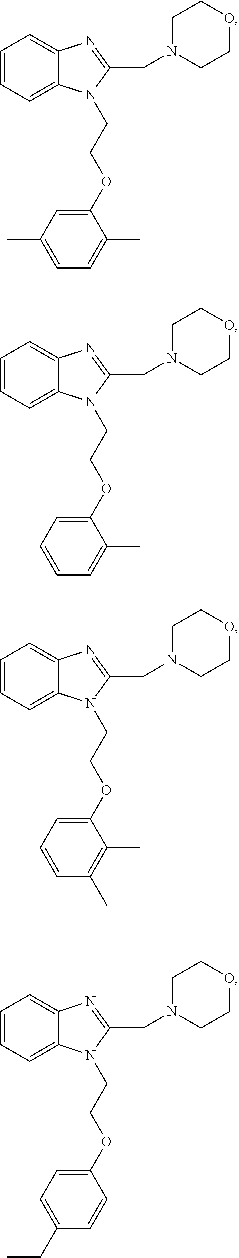 [SMILES: Cc1ccc(C)c(OCCn2c(CN3CCOCC3)nc3ccccc32)c1 ; Cc1ccccc1OCCn1c(CN2CCOCC2)nc2ccccc21 ; Cc1cccc(OCCn2c(CN3CCOCC3)nc3ccccc32)c1C ; CCc1ccc(OCCn2c(CN3CCOCC3)nc3ccccc32)cc1]

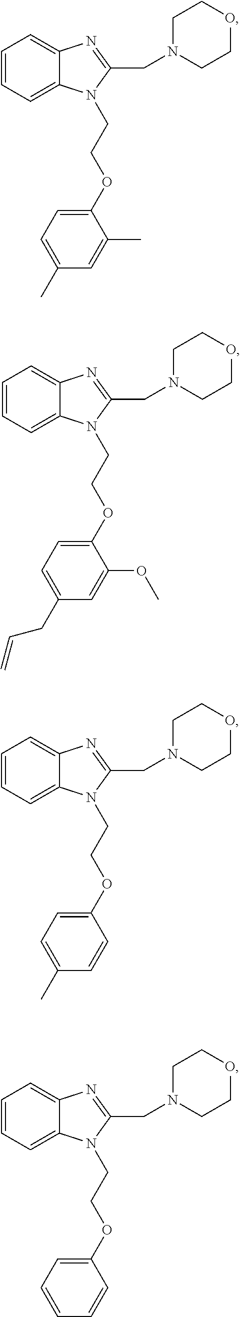 [SMILES: Cc1ccc(OCCn2c(CN3CCOCC3)nc3ccccc32)c(C)c1 ; C=CCc1ccc(OCCn2c(CN3CCOCC3)nc3ccccc32)c(OC)c1 ; Cc1ccc(OCCn2c(CN3CCOCC3)nc3ccccc32)cc1 ; c1ccc(OCCn2c(CN3CCOCC3)nc3ccccc32)cc1]

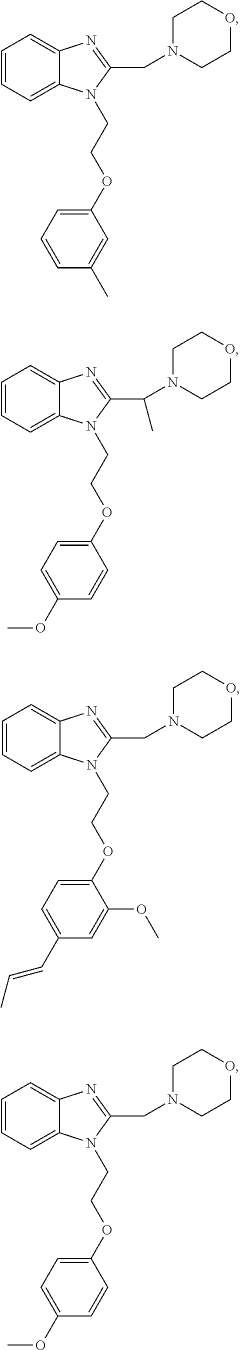 [SMILES: Cc1cccc(OCCn2c(CN3CCOCC3)nc3ccccc32)c1 ; COc1ccc(OCCn2c(C(C)N3CCOCC3)nc3ccccc32)cc1 ; C/C=C/c1ccc(OCCn2c(CN3CCOCC3)nc3ccccc32)c(OC)c1 ; COc1ccc(OCCn2c(CN3CCOCC3)nc3ccccc32)cc1]

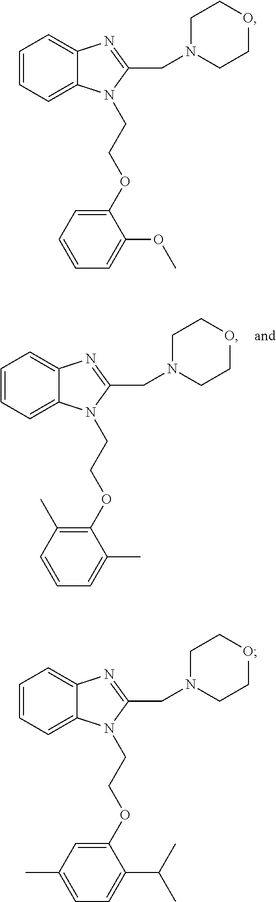 [SMILES: COc1ccccc1OCCn1c(CN2CCOCC2)nc2ccccc21 ; Cc1cccc(C)c1OCCn1c(CN2CCOCC2)nc2ccccc21 ; Cc1ccc(C(C)C)c(OCCn2c(CN3CCOCC3)nc3ccccc32)c1]

or a pharmaceutically acceptable salt thereof.

In another embodiment, the disease or condition that is mediated by elevated persistent sodium current is selected from the group consisting of chronic pain, ocular disorder, multiple sclerosis, and seizure disorder.

In another embodiment, the disease or condition is an ocular disorder selected from the group consisting of age related macular degeneration (AMD) (including wet and dry AMD), geographic atrophy, retinitis pigmentosa, Stargardt's disease cone dystrophy, and pattern dystrophy of the retinal pigmented epithelium, macular edema, retinal detachment, retinal trauma, retinal tumors and retinal diseases associated with said tumors, congenital hypertrophy of the retinal pigmented epithelium, acute posterior multifocal placoid pigment epitheliopathy, optic neuritis, acute retinal pigment epithelitis, optic neuropathies and glaucoma.

In another embodiment, the ocular disorder is selected from the group consisting of age related macular degeneration and geographic atrophy.

In another embodiment, the disease or condition that is mediated by elevated persistent sodium current is multiple sclerosis.

In another embodiment, the disease or condition that is mediated by elevated persistent sodium current is chronic pain selected from the group consisting of neuropathic pain, inflammatory pain, visceral pain, post-operative pain, pain resulting from cancer or cancer treatment, headache pain, irritable bowel syndrome pain, fibromyalgia pain, and pain resulting from diabetic neuropathy.

In another embodiment, the disease or condition that is mediated by elevated persistent sodium current is a central nevous system condition such such as seizure disorders (including epilepsy and chemically induced seizure disorders (e.g., neurotoxins that elevate persistent currents) anxiety, depression and bipolar diseases.

As used herein, the term “pain” refers to all categories of pain and is recognized to include, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g. musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, and combinations thereof.

In another embodiment, the ocular disorder is selected from the group consisting of age related macular degeneration and geographic atrophy.

In another embodiment, the ocular disorder is optic neuritis.

In another embodiment, the disease or condition that is mediated by elevated persistent sodium current is chronic pain.

In another embodiment, the chronic pain of the present invention is selected from the group consisting of neuropathic pain, inflammatory pain such as arthritic pain, visceral pain, post-operative pain, pain resulting from cancer or cancer treatment, headache pain, irritable bowel syndrome pain, fibromyalgia pain, and pain resulting from diabetic neuropathy.

In another embodiment, the disease or condition that is mediated by elevated persistent sodium current is multiple sclerosis.

### General Experimental

Proton NMR spectra were measured at 60 MHz on a Varian T-60A spectrometer or at 300 MHz on a Varian Inova 300 spectrometer. Chemical shifts are expressed in ppm. High pressure liquid chromatography analyses were performed using an Agilent series 1100 HPLC instrument with an Alltech Alltima C₁₈ 5μ, 250×4.6 mm, flow: 1 mL/min at 40° C. Elution was isocratic using a mixture of H₂O, A1 (made up of 700 mL H₂O, 300 mL MeOH, 3 mL Et₃N, and enough phosphoric acid to give a pH of 3.4), and MeOH.

### Compound #1

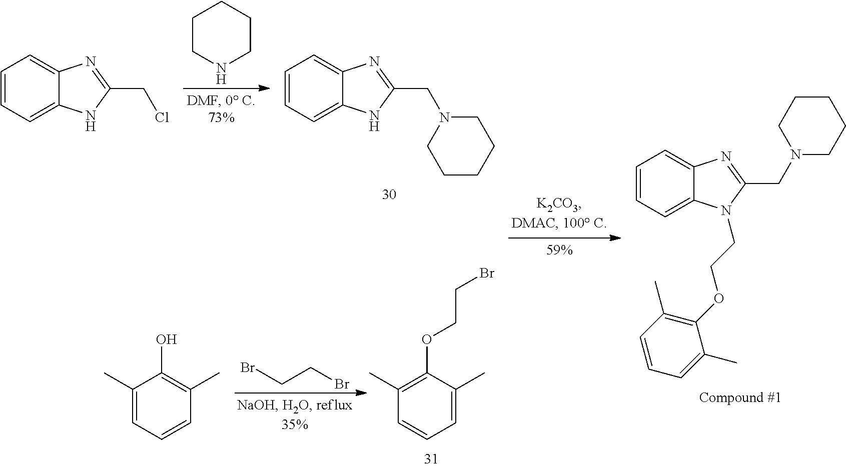

### 2-(Piperidylmethyl)benzimidazole (30)

A 1 L 3-neck flask fitted with a stir-bar, thermometer, addition funnel, and Ar inlet was charged with piperidine (24.0 g, 27.9 mL, 282 mmol) and DMF (50 mL). The resultant solution was cooled to −5° C. in an ice/MeOH bath, and 2-(chloromethyl)benzimidazole (23.5 g, 141 mmol) in DMF (130 mL) was added over 45 min maintaining a temperature below 0° C. The resultant suspension warmed to rt with stirring over 1 hr. The mixture was then cooled in an ice bath, and Et₃N (25 mL) was added followed by H₂O (800 mL). The suspension stirred at rt overnight. The solid was filtered, pressed with rubber dam, and rinsed with H₂O (3×150 mL) and hexanes (150 mL). The material dried to give 22.5 g of 30 as a tan solid (73%). ¹H NMR (60 MHz, CDCl₃): δ 7.6-7.1 (m, 4H), 3.8 (s, 2H), 2.4 (bm, 4H), 1.5 (bs, 6H) ppm. HPLC analysis (15:10:75 H₂O:A1:MeOH) showed a purity of 98% with a retention time of 3.0 min.

### 1-(2,6-Dimethylphenoxy)-2-bromoethane (31)

A 2 L 3-neck flask fitted with a mechanical stirrer, condenser, addition funnel, and Ar inlet was charged with 2,6-dimethylphenol (100 g, 0.820 mol), 1,2-dibromoethane (252 g, 1.34 mol), and H₂O (125 mL). The mixture was heated to reflux, and NaOH (54.0 g, 1.34 mol) in H₂O (500 mL) was added over 30 min. The mixture was then refluxed overnight. After cooling, the phases were separated. The organic phase was diluted with Et₂O (500 mL), then washed with 1 M NaOH (2×200 mL) and brine (200 mL). The solution was concentrated to a yellow oil that was washed with 1M NaOH (3×130 mL). The aqueous phase was back-extracted with Et₂O (100 mL). The combined organic phases were then diluted with Et₂O (300 mL) and washed with brine (100 mL). The organic phase was dried over MgSO₄ and concentrated to a yellow oil. The oil was further concentrated with stirring under high vacuum to give 95 g of an oil which was then vacuum distilled. The fraction containing product distilled at 65-70° C. at 0.4 mm to give 69.9 g oil. The resultant material was filtered through silica gel (120 g) and Na₂SO₄ (30 g) with hexanes (2×250 mL). The solution was concentrated in vacuo and stirred under high vacuum to give 65 g of 31 as a colorless oil (35%). ¹H NMR (60 MHz, CDCl₃): δ 7.1-6.9 (m, 3H), 4.3-4.0 (m, 2H), 3.8-3.5 (m, 2H), 2.4 (s, 6H) ppm. HPLC analysis (0:10:90 H₂O:A1:MeOH) showed a purity of 98% with a retention time of 4.7 min.

### 1-[2-(2,6-Dimethylphenoxy)ethyl]-2-piperidin-1-ylmethyl-1H-benzimidazole (Compound #1)

A 500 mL 3-neck flask fitted with a stir-bar, addition funnel, and Ar inlet was charged with benzimidazole 30 (20.0 g, 92.9 mmol), K₂CO₃ (25.7 g, 186 mmol), and DMAC (200 mL). Bromide 31 (21.3 g, 92.9 mmol) was then added, and the mixture was heated in a 100° C. bath overnight. After cooling to rt, the solids were filtered off and rinsed with EtOAc (500 mL). The filtrate was washed with H₂O (2×250 mL), saturated aqueous NH₄Cl (3×250 mL), and brine (250 mL). The organic phase was filtered through phase separation paper and concentrated in vacuo. The resultant material was reconcentrated from CH₂Cl₂ (50 mL) and dried under high vacuum to 32.8 g of an orange glass. The crude material was chromatographed on silica gel, with the product eluting at 10-25% EtOAc in hexanes. The fractions were concentrated in vacuo, and the ensuing crystals were filtered with the aid of hexanes (20 mL) and rinsed with hexanes (10 mL). The material was dried to give 19.8 g of Compound #1 as pale yellow crystals (59%). ¹H NMR (300 MHz, CDCl₃): δ 7.76 (m, 1H), 7.45 (m, 1H), 7.26 (m, 2H), 7.00-6.85 (m 3H), 4.80 (t, 2H), 4.16 (t, 2H), 3.89 (s, 2H), 2.48 (bs, 4H), 2.03 (s, 6H), 1.60-1.40 (bm, 6H) ppm. HPLC analysis (5:10:85 H₂O:A1:MeOH) showed a purity of greater than 99% with a retention time of 5.9 min.

### Compound #2

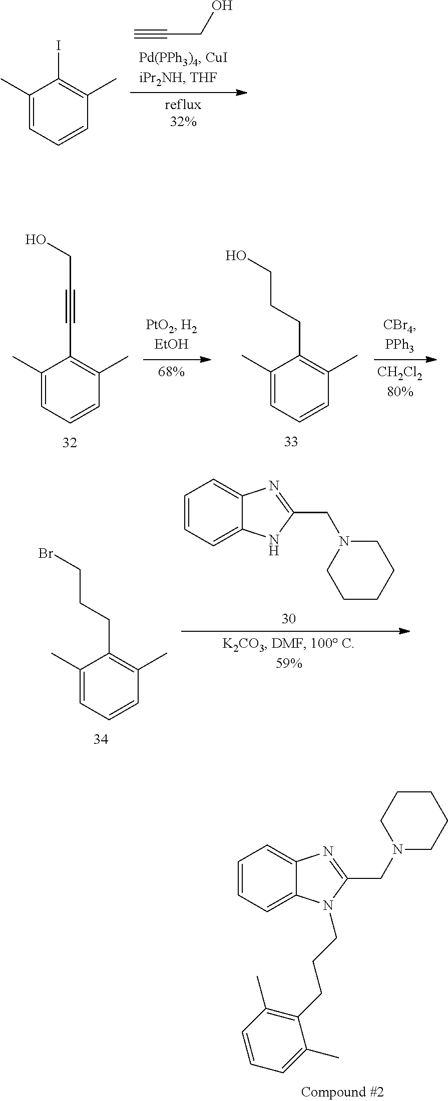

### 3-(2,6-Dimethylphenyl)prop-2-yn-1-ol (32)

A 500 mL 3-neck flask fitted with a stir-bar, condenser, and Ar inlet was charged with 2,6-dimethyliodobenzene (21.6 g, 93.1 mmol), propargyl alcohol (6.78 g, 7.15 mL, 121 mmol), iPr₂NH (100 mL), and THF (100 mL). The resultant solution was sparged with Ar for 10 min, then Pd(PPh₃)₄ (1.00 g, 0.93 mmol) and CuI (350 mg, 1.86 mmol) were added. This mixture was heated at 50° C. for 1.5 hr. HPLC analysis showed the reaction to be incomplete. 8 mL more propargyl alcohol was added and the mixture was heated at reflux overnight. The reaction was still incomplete by HPLC analysis. Another 10 mL propargyl alcohol, 500 mg Pd(PPh₃)₄, and 200 mg CuI were added, and the mixture was refluxed for 6 hr. Another 8 mL portion of propargyl alcohol was then added, and the reaction was continued at reflux overnight. The mixture was then cooled, and the solids were filtered off and rinsed with EtOAc (200 mL). The filtrate was concentrated to a dark oil and partitioned with CH₂Cl₂ (500 mL) and saturated NH₄Cl (250 mL). The organic phase was washed with saturated aqueous NH₄Cl (250 mL) and brine (200 mL) and filtered through phase separation paper. The solution was then filtered through a plug of silica gel. The silica gel was rinsed with CH₂Cl₂ (1.2 L). The combined filtrate was concentrated to 8.5 g of a brown oil. The crude material was chromatographed on silica gel with the product eluting with 10-12% EtOAc in hexanes. The combined fractions were concentrated in vacuo, taken up in CH₂Cl₂ (200 mL), and washed with 5% aqueous Na₂S₂O₃ (200 mL) then brine (200 mL). The solution was filtered through phase separation paper and concentrated in vacuo to give 5.0 g of 32 as an orange solid (32%). ¹H NMR (60 MHz, CDCl₃): δ 7.3-6.8 (m, 3H), 4.5 (s, 2H), 2.8 (s, 1H), 2.4 (s, 6H) ppm. HPLC analysis (5:10:85 H₂O:A1:MeOH) showed a purity of greater than 99% with a retention time of 3.7 min.

### 3-(2,6-Dimethylphenyl)propan-1-ol (33)

A 250 mL hydrogenation vessel was charged with phenylacetylene 32 (5.0 g, 31.2 mmol) and EtOH (50 mL). To the resultant solution was added PtO₂ (300 mg). The mixture was hydrogenated on a Parr shaker at 49 psi overnight. The reduction was incomplete as monitored by HPLC. The catalyst was filtered off on glass-fiber paper, and 300 mg fresh PtO₂ were added. The mixture hydrogenated at 60 psi overnight to completion. The catalyst was filtered off on glass fiber paper, and the filtrate was concentrated to a brown oil. The material was filtered through a plug of silica gel with CH₂Cl₂ (3×75 mL) and concentrated to in vacuo 3.55 g of a yellow oil. However, the material was now determined to be ˜1:1 mixture of product and olefin. The oil was then taken up in EtOH (100 mL), and PtO₂ (300 mg) was added. The mixture was again hydrogenated at 60 psi overnight. The catalyst was filtered off on glass fiber paper, and the filtrate concentrated in vacuo to give 3.5 g of 33 as a light yellow oil (68%). ¹H NMR (60 MHz, CDCl₃): δ 6.9 (s, 3H), 3.6 (t, 2H), 2.8 (s, 1H), 2.8-2.5 (m, 2H), 2.3 (s, 6H), 1.9-1.4 (m, 2H) ppm. HPLC analysis (5:10:85 H₂O:A1:MeOH) showed a purity of 97% with retention time of 3.8 min. (The reduction was also attempted using Pd/C without success.)

### 2-(3-Bromopropyl)-1,3-dimethylbenzene (34)

A 200 mL 3-neck flask fitted with a stir-bar and Ar inlet was charged with alcohol 33 (3.40 g, 20.7 mmol) and CH₂Cl₂ (20 mL). The resultant solution was immersed in an ice bath. CBr₄ (8.93 g, 26.9 mmol) in CH₂Cl₂ (15 mL) was added followed by PPh₃ (7.06 g, 26.9 mmol) in CH₂Cl₂ (15 mL). The mixture was stirred in an ice bath for 10 min, then warmed to rt over 3 hr. The mixture was diluted with CH₂Cl₂ (75 mL) and washed with half-saturated NaHCO₃ (2×100 mL) and brine (100 mL). The solution was filtered through phase separation paper and concentrated in vacuo to a solid/oil mix. The material was triturated with a small amount of hexanes overnight, then filtered. The filtrate was concentrated to 7.8 g of a yellow oil. This crude oil was chromatographed on silica gel with hexanes. The product fractions were concentrated to give 4.0 g of 34 as a yellow oil with a purity of 80% by HPLC (80%). The material was taken on as is. ¹H NMR (60 MHz, CDCl₃): δ 6.9 (s, 3H), 3.5 (t, 2H), 2.9-2.6 (m, 2H), 2.3 (s, 6H), 2.2-1.8 (m, 2H) ppm. HPLC analysis (0:10:90 H₂O:A1:MeOH) showed a purity of 80% with a retention time of 6.0 min.

### 1-[3-(2,6-Dimethylphenyl)-propyl]-2-piperidin-1-ylmethyl-1H-benzimidazole (Compound #2)

A 50 mL flask fitted with a stir-bar and septum with an Ar inlet was charged with benzimidazole 30 (1.00 g, 4.64 mmol), bromide 34 (1.32 g, 5.80 mmol), K₂CO₃ (963 mg, 6.97 mmol), and DMF (15 mL). The mixture was heated at 100° C. for 2 hr. The bromide was consumed before the benzimidazole, as monitored by HPLC. Consequently ˜800 mg more bromide were added, and the mixture was heated at 100° C. overnight. The mixture was cooled, and the solids were filtered off and rinsed with EtOAc (50 mL). The filtrate was washed with saturated aqueous NH₄Cl (2×50 mL), H₂O (2×50 mL), and brine (50 mL), filtered through phase separation paper, and concentrated in vacuo to a yellow solid. The crude material was chromatographed on a 25+M Biotage MPLC silica gel column eluting with 2-50% EtOAc in hexanes. The product eluted with 25-50% EtOAc in hexanes. The product fractions were concentrated to a white solid which was collected with hexanes to give 800 mg. A second crop of 150 mg was collected from the supernatant. The combined material was dried to give 950 mg of Compound #2 as a white solid (59%). ¹H NMR (300 MHz, CDCl₃): δ 7.78-7.72 (m, 1H), 7.36-7.30 (m, 1H), 7.30-7.21 (m, 2H), 7.02 (s, 3H), 4.43 (t, 2H), 3.75 (s, 2H), 2.78-2.70 (m, 2H), 2.43 (bs, 4H), 2.29 (s, 6H), 2.15-2.02 (m, 2H), 1.60-1.40 (bm, 6H) ppm. HPLC analysis (0:10:90 H₂O:A1:MeOH) showed a purity of 97% with a retention time of 5.7 min.

### Compound #s 3 and 4

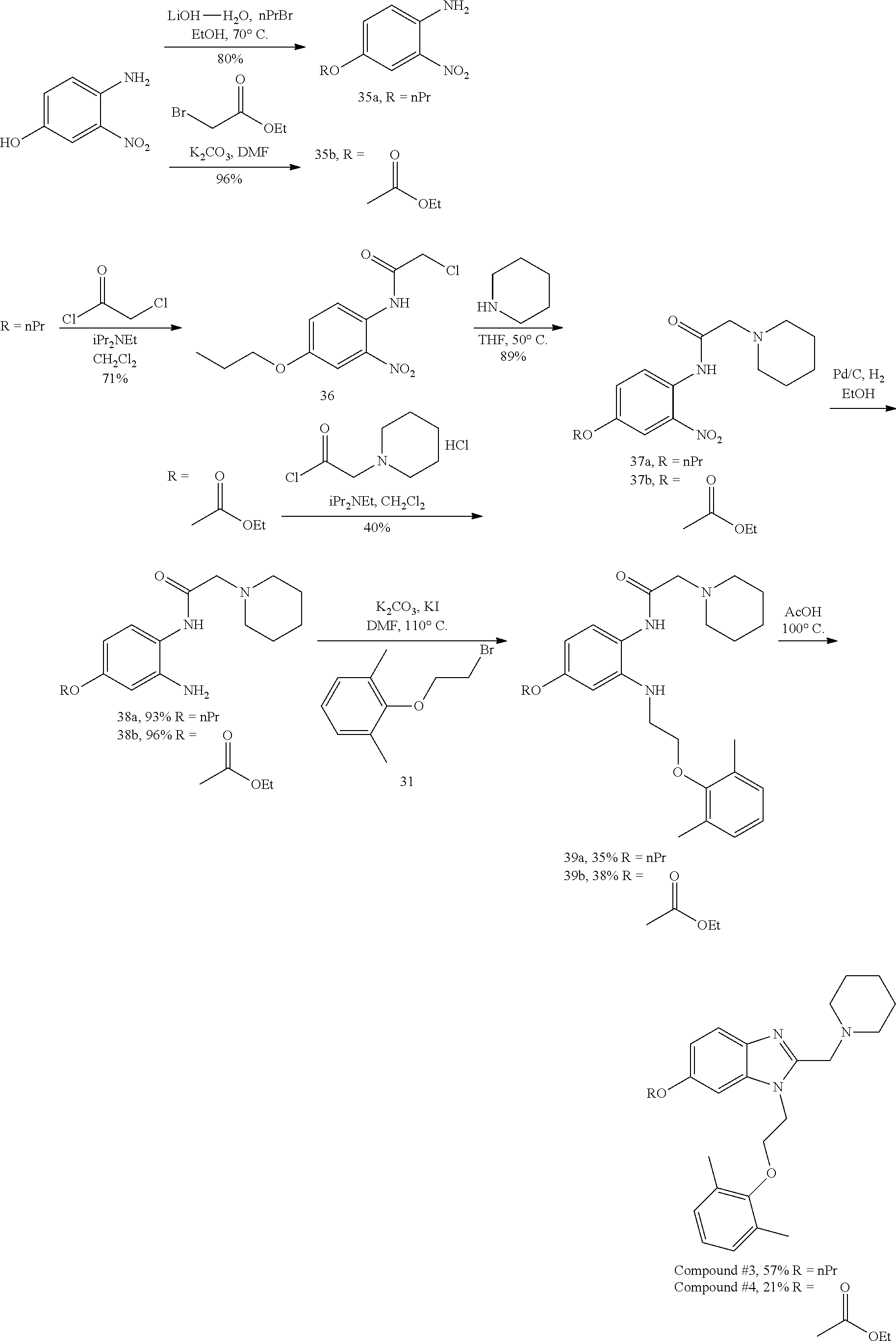

### 2-Nitro-4-propoxyaniline (35a)

A 250 mL 3-neck flask fitted with a stir-bar and an Ar inlet was charged with 4-amino-3-nitrophenol (5.00 g, 32.4 mmol), n-propyl bromide (5.59 g, 4.13 mL, 45.4 mmol), LiOH.H₂O (2.72 g, 64.8 mmol), and EtOH (50 mL). The mixture was heated at 70° C. overnight. Another 0.5 mL n-propyl bromide was added and the mixture was further heated for 5 hr. An additional 500 mg LiOH.H₂O and 0.5 mL n-propyl bromide were added and the mixture was heated overnight. After cooling, the solution was partitioned with H₂O (100 mL) and EtOAc (150 mL). The organic phase was washed with 1% aqueous LiOH (2×100 mL), H₂O (100 mL), and brine (100 mL), filtered through phase separation paper, and concentrated in vacuo to give 6.3 g of a red solid. The crude material was triturated with hexanes:EtOAc 10:1 (30 mL), filtered, and pressed with rubber dam. The solid was rinsed with hexanes (2×10 mL) to give 5.1 g 35a as a red solid (80%). HPLC analysis (15:10:75 H₂O:A1:MeOH) showed a purity of 97% with a retention time of 5.3 min.

### 2-Chloro-N-(2-nitro-4-propoxyphenyl)acetamide (36)

A 500 mL 3-neck flask fitted with a stir-bar, addition funnel, and an Ar inlet was charged with aniline 35a (5.65 g, 28.8 mmol) and CH₂Cl₂ (100 mL). iPr₂NEt (11.1 g, 15.0 mL, 86.4 mmol) was added followed by chloroacetyl chloride (6.51 g, 4.59 mL, 57.6 mmol) over 15 min. The mixture was stirred 4 hr at rt. Another 0.5 mL more chloroacetyl chloride was added, and the mixture was stirred 2 hr to completion as monitored by HPLC. The mixture was diluted with CH₂Cl₂ (100 mL) and washed with saturated aqueous NH₄Cl (100 mL), saturated NaHCO₃ (100 mL), and brine (100 mL). The mixture was then filtered through phase separation paper and concentrated in vacuo to a dark oil. The oil was filtered through a plug of silica gel with CH₂Cl₂ (400 mL) and concentrated to 8.0 g of a dark red solid. The crude solid was chromatographed on silica gel. The product eluted with 10-15% EtOAc in hexanes to give 3.1 g of an orange solid. Later fractions were rechromatographed, eluting product with 7-8% EtOAc in hexanes providing additional product. The combined material yielded 4.8 g of 36 as an orange solid (61%). ¹H NMR (60 MHz, CDCl₃): δ 8.5 (d, 1H), 7.6 (d, 1H), 7.2 (dd, 1H), 4.2 (s, 1H), 4.0 (t, 2H), 2.1-1.6 (m, 2H), 1.1 (t, 3H) ppm. HPLC analysis (5:10:85 H₂O:A1:MeOH) showed a purity of 97% with a retention time of 4.2 min.

### N-(2-Nitro-4-propoxyphenyl)-2-piperidylacetamide (37a)

A 250 mL 3-neck flask fitted with a stir-bar and an Ar inlet was charged with chloride 36 (4.75 g, 17.4 mmol) and THF (50 mL). Piperidine (3.26 g, 3.79 mL, 38.3 mmol) was added, and the mixture was heated at 50° C. for 4 hr. Another 1 mL piperidine was then added, and the solution was heated at 50° C. another 2 hr. The mixture was cooled in an ice bath, and the solids were filtered off and rinsed with THF (2×25 mL). The filtrate was concentrated to an orange oil. This oil was taken up in EtOAc (100 mL) and washed with H₂O (75 mL), saturated aqueous NH₄Cl (75 mL), saturated NaHCO₃ (75 mL), and brine (50 mL). The solution was filtered through phase separation paper and concentrated in vacuo to give 5.67 g of 37a as an orange oil (100%). ¹H NMR (60 MHz, CDCl₃): δ 8.7 (d, 1H), 7.7 (d, 1H), 7.2 (dd, 1H), 4.0 (t, 2H), 3.2 (s, 2H), 2.6-2.4 (m, 4H), 2.1-1.3 (m, 8H), 1.0 (t, 3H) ppm. HPLC analysis (5:10:85 H₂O:A1:MeOH) showed a purity of 98% with a retention time of 5.3 min.

### N-(2-Amino-4-propoxyphenyl)-2-piperidylacetamide (38a)

A 500 mL hydrogenation vessel was charged with nitroarene 37a (5.60 g, 17.4 mmol) and EtOH (60 mL). 10% Pd/C (400 mg) was added to the resultant solution, and the mixture was hydrogenated on a Parr shaker at 60 psi for 4 hr. The reaction had reached completion as monitored by HPLC. The catalyst was then filtered off on glass fiber paper and rinsed with EtOH (2×15 mL). The filtrate was concentrated in vacuo to give 4.74 g of 38a as an off-white solid (93%). ¹H NMR (60 MHz, CDCl₃): δ 7.0 (d, 1H), 6.4 (d, 1H), 6.4 (dd, 1H), 3.9 (t, 2H), 3.9 (bs, 2H), 3.1 (s, 1H), 2.7-2.5 (m, 4H), 1.9-1.5 (m, 8H), 1.1 (t, 3H) ppm. HPLC analysis (5:10:85 H₂O:A1:MeOH) showed a purity of 99% with a retention time of 2.6 min.

### N-(2-{[2-(2,6-Dimethylphenoxy)ethyl]amino}-4-propoxyphenyl)-2-piperidylacetamide (39a)

A 100 mL 3-neck flask fitted with a stir-bar, condenser, and an Ar inlet was charged with aniline 38a (3.00 g, 10.3 mmol), bromide 31 (2.96 g, 12.9 mmol), K₂CO₃ (2.14 g, 15.5 mmol), KI (166 mg, 1.00 mmol), and DMF (30 mL). The suspension was heated in a 110° C. bath overnight. Another 2 g of bromide 31 were added, and the mixture was heated at 110° C. another 4 hr then cooled to rt overnight. The solids were filtered off and rinsed with EtOAc (100 mL). The filtrate was washed with saturated aqueous NH₄Cl (2×60 mL), H₂O (2×60 mL) and brine (50 mL), filtered through phase separation paper, and concentrated to 5.8 g of an oil. The oil was partitioned with EtOAc (60 mL) and 5% aqueous citric acid. The organic phase was extracted with 5% aqueous citric acid (3×60 mL). All phases were left for 2 days forming crystals. All phases were combined, and the crystals were filtered. The crystals were then suspended in saturated NaHCO₃ (50 mL) and extracted with EtOAc (50 mL). The organic phase was washed with saturated NaHCO₃ (50 mL) and brine (50 mL), filtered through phase separation paper, and concentrated to give 1.6 g of 39a as a light brown, crystalline solid (35%). ¹H NMR (300 MHz, CDCl₃): δ 8.86 (bs, 1H), 7.27 (s, 1H), 7.15 (d, 1H), 7.06-6.92 (m, 3H), 6.38 (d, 1H), 6.34 (dd, 1H), 4.63 (bt, 1H), 4.03 (t, 2H), 3.93 (t, 3H), 3.52 (qt, 2H), 3.13 (s, 2H), 2.62-2.55 (m, 4H), 2.29 (s, 6H), 1.82 (sept, 2H), 1.69-1.57 (m, 4H), 1.52-1.41 (m, 2H), 1.04 (t, 3H) ppm. HPLC analysis (0:10:90 H₂O:A1:MeOH) showed a purity of 98% with a retention time of 3.3 min.

### 1-[2-(2,6-Dimethylphenoxy)-ethyl]-2-piperidin-1-ylmethyl-6-propoxy-1H-benzimidazole (Compound #3)

A 100 mL flask fitted with a stir-bar, condenser, and an Ar inlet was charged with amine 39a (1.50 g, 3.41 mmol) and AcOH (20 mL). The resultant solution was heated at 100° C. for 6 hr then cooled to rt overnight. The mixture was concentrated to an orange oil and taken up in EtOAc (50 mL). The solution was washed with saturated NaHCO₃ (2×25 mL) and brine (25 mL), filtered through phase separation paper, and concentrated to a brown solid. The crude solid was triturated with hexanes:EtOAc 10:1 (15 mL) and filtered. The solid was rinsed with hexanes (2×8 mL) and hexanes:EtOAc 1:1 (20 mL). The filtrate was concentrated in vacuo to a tan solid and triturated with 20:1 hexanes:EtOAc (10 mL). The solid was filtered and rinsed with hexanes (2×5 mL) to give 800 mg of Compound #3 as a tan solid (57%). ¹H NMR (300 MHz, CDCl₃): δ 7.62 (d, 1H), 7.00-6.88 (m, 5H), 4.76 (bt, 2H), 4.16 (t, 2H), 3.97 (t, 2H), 3.84 (bs, 2H), 2.45 (bs, 4H), 2.03 (s, 6H), 2.84 (sept, 2H), 1.64-1.38 (m, 6H), 1.06 (t, 3H) ppm. HPLC analysis (0:10:90 H₂O:A1:MeOH) showed a purity of 97% with a retention time of 4.5 min.

### Ethyl 2-(4-amino-3-nitrophenoxy)acetate (35b)

A 200 mL 3-neck flask fitted with a stir-bar and an Ar inlet was charged with 4-amino-3-nitrophenol (5.00 g, 32.4 mmol), K₂CO₃ (4.93 g, 35.6 mmol), and DMF (50 mL). To the suspension was added ethyl bromoacetate (5.68 g, 3.77 mL, 34.0 mmol). The mixture stirred 2.5 hr to completion as monitored by HPLC. The solids were filtered off and rinsed with EtOAc (150 mL). The filtrate was washed with saturated aqueous NH₄Cl (2×100 mL), H₂O (2×100 mL) and brine (100 mL), filtered through phase separation paper, and concentrated in vacuo to give 8.6 g of a red solid. The solid was triturated with hexanes:EtOAc 3:1 (20 mL). The solid was then filtered, pressed with rubber dam, and rinsed with the same solvent mix (2×25 mL) to give 7.3 g of 35b as a bright orange solid (94%). ¹H NMR (60 MHz, CDCl₃): δ 7.4 (d, 1H), 7.1 (dd, 1H), 6.7 (d, 1H), 6.1 (bs, 2H), 4.5 (s, 2H), 4.3 (qt, 2H), 1.3 (t, 3H) ppm. HPLC analysis (15:10:75 H₂O:A1:MeOH) showed a purity of 98% with a retention time of 3.5 min.

### Ethyl 2-[3-nitro-4-(2-piperidylacetylamino)phenoxy]acetate (37b)

A 500 mL 3-neck flask fitted with a stir-bar, addition funnel, and an Ar inlet was charged with aniline 35b (5.45 g, 22.7 mmol) and CH₂Cl₂ (100 mL). The resultant solution was cooled in an ice bath and iPr₂NEt was added followed by piperidylacetyl chloride hydrochloride (5.40 g, 27.3 mmol) in CH₂Cl₂ over 15 min. The mixture was stirred at rt overnight. Another 7.0 g of the acid chloride was added and the solution was stirred overnight again. The mixture was partitioned with H₂O (125 mL), and the organic phase was washed with saturated NaHCO₃ (125 mL), H₂O (125 mL), and brine (100 mL). The solution was filtered through phase separation paper and concentrated in vacuo to 11.0 g of a dark red oil. The crude material was chromatographed on silica gel, eluting the product with 15% EtOAc in hexanes. The product fractions were concentrated to an orange solid which was triturated with hexanes:EtOAc 30:1 (30 mL). The solid was filtered to give 2.1 g of nitroarene 35b as a yellow solid (25%). ¹H NMR (60 MHz, CDCl₃): δ 8.7 (d, 1H), 7.6 (d, 1H), 7.2 (dd, 1H), 4.6 (s, 2H), 4.2 (qt, 2H), 3.1 (s, 2H), 2.7-2.4 (m, 4H), 1.9-1.4 (m, 6H), 1.3 (t, 3H) ppm. HPLC analysis (15:10:75 H₂O:A1:MeOH) showed a purity of 96% with a retention time of 3.8 min.

### Ethyl 2-[3-Amino-4-(2-piperidylacetylamino)phenoxy]acetate (38b)

A 250 mL hydrogenation vessel was charged with nitroarene 37b (2.10 g, 5.75 mmol) and EtOH (25 mL). 10% Pd/C (250 mg) was added to the resultant solution, and the mixture was hydrogenated on a Parr shaker at 43 psi for 3.5 hr. The reaction was then complete as monitored by HPLC. The catalyst was filtered off on glass fiber paper and rinsed with EtOH (20 mL). The filtrate was concentrated in vacuo to a tan solid, which was triturated with hexanes:EtOAc 20:1 (20 mL). The solid was then filtered to give 1.85 g of 38b as an off-white solid (96%). ¹H NMR (60 MHz, CDCl₃): δ 9.0 (bs, 1H), 7.3 (d, 1H), 6.6 (d, 1H), 6.5 (dd, 1H), 4.7 (s, 2H), 4.4 (qt, 2H), 4.1 (bs, 2H), 3.2 (s, 2H), 2.8-2.5 (m, 4H), 1.8-1.4 (m, 6H), 1.4 (t, 3H) ppm. HPLC analysis (15:10:75 H₂O:A1:MeOH) showed a purity of 97% with a retention time of 2.5 min.

### Ethyl 2-(3-{[2-(2,6-dimethylphenoxy)ethyl]amino}-4-(2-piperidylacetylamino)-phenoxy)acetate (39b)

A 100 mL 3-neck flask fitted with a stir-bar, condenser, and an Ar inlet was charged with aniline 38b (1.80 g, 5.37 mmol), bromide 31 (1.29 g, 5.64 mmol), K₂CO₃ (1.11 g, 8.06 mmol), KI (100 mg, 0.60 mmol), and DMF (20 mL). The suspension was heated in a 110° C. bath overnight. Another 1 g of bromide 31 was added, and the mixture was heated at 110° C. for another 3 hr. The mixture was cooled, and the solids were filtered off and rinsed with EtOAc (100 mL). The filtrate was washed with saturated aqueous NH₄Cl (2×50 mL), H₂O (2×50 mL), and brine (50 mL), filtered through phase separation paper, and concentrated in vacuo to give 5.0 g of a brown oil. The oil was chromatographed on silica gel eluting the product with 40-50% EtOAc in hexanes. The product fractions were concentrated in vacuo to give 1.0 g of 39b as an orange solid (38%). ¹H NMR (300 MHz, CDCl₃): δ 8.86 (s, 1H), 7.17 (d, 1H), 7.05-6.91 (m, 4H), 6.43 (d, 1H), 6.28 (dd, 1H), 4.61 (s, 2H), 4.27 (qt, 2H), 4.02 (t, 2H), 3.49 (qt, 2H), 3.12 (s, 2H), 2.57 (bs, 4H), 2.26 (s, 6H), 1.68-1.57 (m, 4H), 1.52-1.40 (m, 2H), 1.32 (t, 3H) ppm. HPLC analysis (0:10:90 H₂O:A1:MeOH) showed a purity of 85% with a retention time of 3.7 min.

### {1-[2-(2,6-Dimethylphenoxy)-ethyl]-2-piperidin-1-ylmethyl-1H-benzimidazol-5-yloxy}acetic acid ethyl ester (Compound #4)

A 100 mL flask fitted with a stir-bar, condenser, and an Ar inlet was charged with amine 39b (950 mg, 1.96 mmol) and AcOH (12 mL). The resultant solution was heated at 100° C. for 6 hr, then cooled to rt overnight. The mixture was concentrated in vacuo to an orange oil and taken up in EtOAc (25 mL). The solution was washed with saturated NaHCO₃ (3×10 mL) and brine (10 mL), filtered through phase separation paper, and concentrated to give 840 mg of an orange oil. The crude material was chromatographed with several Biotage silica gel columns to obtain sufficient purity. The product eluted through the first column with 30-55% EtOAc in hexanes to obtain a purity of ˜75%. Elution through 3 subsequent columns with 1-3% EtOH in hexanes gave 195 mg of Compound #4 as a yellow oil (21%). ¹H NMR (300 MHz, CDCl₃): δ 7.64 (d, 1H), 7.03-6.86 (m, 5H), 4.72 (m, 2H), 4.66 (s, 2H), 4.17 (qt, 2H), 4.12 (m, 2H), 3.83 (s, 2H), 2.45 (bs, 4H), 2.00 (s, 6H), 1.56-1.37 (m 6H), 1.24 (t, 3H) ppm. HPLC analysis (0:10:90 H₂O:A1:MeOH) showed a purity of 95% with a retention time of 3.7 min.

### Compound #5

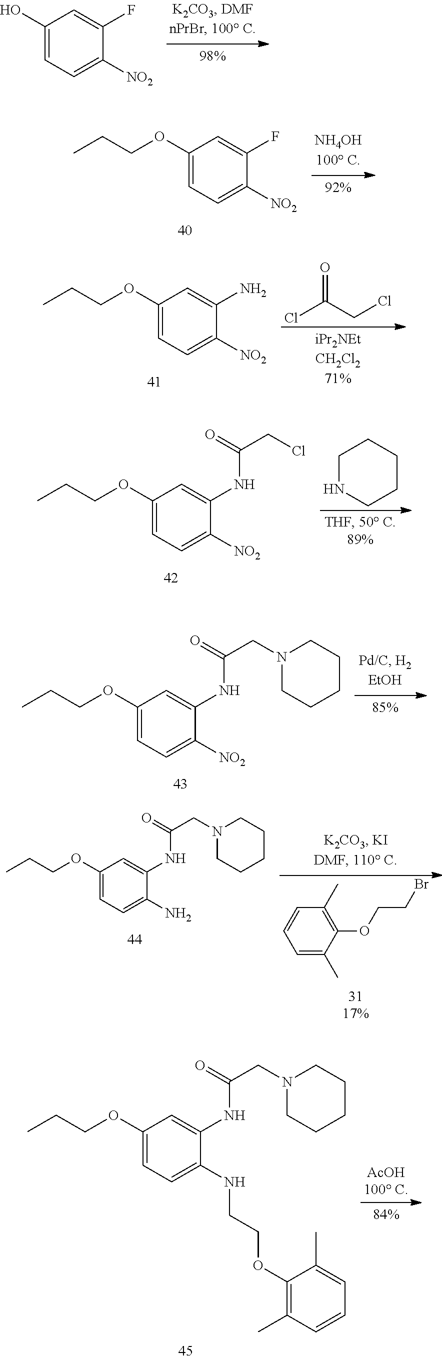

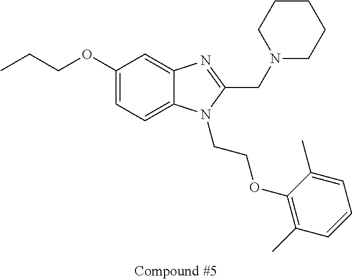 [SMILES: CCCOc1ccc2c(c1)nc(CN1CCCCC1)n2CCOc1c(C)cccc1C]

### 2-Fluoro-1-nitro-4-propoxybenzene (40)

A 250 mL flask fitted with a stir-bar and septum with an Ar inlet was charged with 3-fluoro-4-nitrophenol (5.00 g, 31.8 mmol), n-propyl bromide (5.09 g, 3.76 mL, 41.4 mmol), and DMF (50 mL). To the resultant suspension was added K₂CO₃ (6.59 g, 47.7 mmol), and the mixture was heated in a 100° C. bath for 75 min. The mixture was then cooled, and the solids were filtered off and rinsed with EtOAc (200 mL). The filtrate was washed with H₂O (4×125 mL) and brine (100 mL), filtered through phase separation paper, and concentrated to give 6.3 g of an orange oil. After stirring under high vacuum, 6.2 g of 40 was yielded as an orange oil (98%). HPLC analysis showed a 15% impurity, however the material was carried on as is. ¹H NMR (60 MHz, CDCl₃): δ 8.0 (m, 1H), 6.8 (m, 1H), 6.6 (m, 1H), 4.0 (t, 2H), 1.8 (sept, 2H), 1.0 (t, 3H) ppm. HPLC analysis (15:10:75 H₂O:A1:MeOH) showed a purity of 82% with a retention time of 6.5 min.

### 2-Nitro-5-propoxyaniline (41)

A 350 mL pressure vessel was charged with nitroarene 40 (6.20 g, 31.1 mmol) and concentrated aqueous NH₄OH (100 mL). The vessel was sealed, and the suspension was heated in a 105° C. bath overnight. After cooling the solid was filtered, pressed with rubber dam, and rinsed with H₂O (2×20 mL). The solid was triturated in 1M HCl (60 mL), filtered, pressed with rubber dam, and rinsed with H₂O (2×20 mL). The material was then dried to give 5.6 g of 41 as a red solid (92%). ¹H NMR (60 MHz, CDCl₃): δ 8.0 (d, 1H), 6.5-6.1 (m, 4H), 3.9 (t, 2H), 1.8 (sept, 2H), 1.1 (t, 3H) ppm. HPLC analysis (15:10:75 H₂O:A1:MeOH) showed a purity of 96% with a retention time of 5.2 min.

### 2-Chloro-N-(2-nitro-5-propoxyphenyl)acetamide (42)

A 500 mL 3-neck flask fitted with a stir-bar, addition funnel, and an Ar inlet was charged with aniline 41 (5.50 g, 28.0 mmol) and CH₂Cl₂ (100 mL). The resultant solution was cooled in an ice bath, and iPr₂NEt (10.8 g, 14.6 mL, 84.0 mmol) was added followed by chloroacetyl chloride (3.48 g, 2.45 mL, 30.8 mmol) in CH₂Cl₂ (50 mL) over 30 min. The mixture was then stirred at rt for 6 hr. Another 5 mL of chloroacetyl chloride was added, and the mixture was stirred overnight. The solution was diluted with CH₂Cl₂ (100 mL) and washed with saturated aqueous NH₄Cl (100 mL), saturated NaHCO₃ (100 mL) and brine (100 mL). The organic phase was then filtered through phase separation paper and concentrated to give 12 g of a dark, sticky solid. The material was filtered through a plug of silica gel with CH₂Cl₂ (300 mL) and concentrated to give 10 g of a brown solid. The solid was triturated with hexanes:EtOAc 2:1 (30 mL), filtered, and rinsed with the same solvent mix (15 mL). The material dried to give 5.4 g of 42 as a tan solid (71%). ¹H NMR (60 MHz, CDCl₃): δ 8.4 (d, 1H), 8.3 (d, 1H), 6.8 (dd, 1H), 4.2 (s, 2H), 4.1 (t, 2H), 1.8 (sept, 2H), 1.1 (t, 3H) ppm. HPLC analysis (5:10:85 H₂O:A1:MeOH) showed a purity of 95% with a retention time of 4.7 min.

### N-(2-Nitro-5-propoxyphenyl)-2-piperidylacetamide (43)

A 250 mL 3-neck flask fitted with a stir-bar and an Ar inlet was charged with chloride 42 (5.30 g, 19.4 mmol) and THF (55 mL). The resultant solution was cooled in an ice bath and piperidine (3.64 g, 42.8 mmol) was slowly added. The mixture warmed to rt over 1 hr and was then heated at 55° C. overnight. The mixture was then cooled in an ice bath. The solids were filtered off and rinsed with THF (2×25 mL). The filtrate was concentrated in vacuo to a solid and partitioned with EtOAc (100 mL) and H₂O (75 mL). The organic phase was washed with saturated aqueous NH₄Cl (75 mL), saturated NaHCO₃ (75 mL), and brine (75 mL), filtered through phase separation paper, and concentrated to give 5.8 g of a tan solid. The solid was filtered through a plug of silica gel with CH₂Cl₂ (600 mL) and concentrated in vacuo to give 5.5 g of 43 as a bright yellow solid (89%). ¹H NMR (60 MHz, CDCl₃): δ 8.4 (d, 1H), 8.2 (d, 1H), 6.6 (dd, 1H), 4.0 (t, 2H), 3.2 (s, 2H), 2.7-2.4 (m, 4H), 2.0-1.3 (m, 8H), 1.1 (t, 3H) ppm. HPLC analysis (5:10:85 H₂O:A1:MeOH) showed a purity of 99% with a retention time of 7.2 min.

### N-(2-Amino-5-propoxyphenyl)-2-piperidylacetamide (44)

A 500 mL hydrogenation vessel was charged with piperidine 43 (5.50 g, 17.1 mmol) and EtOH (60 mL). 10% Pd/C (500 mg) was added to the resultant suspension, and the mixture was hydrogenated on a Parr shaker at 57 psi for 3 hr. The catalyst was filtered off on glass fiber paper and rinsed with EtOH (50 mL). The filtrate was concentrated in vacuo to a solid. This solid was collected with hexanes:EtOAc 10:1 (30 mL) and dried to give 4.15 g of aniline 44 as an off-white solid (85%). ¹H NMR (60 MHz, CDCl₃): δ 7.1 (d, 1H), 6.6 (d, 1H), 6.6 (dd, 1H), 3.8 (t, 2H), 3.4 (bs, 2H), 3.0 (s, 2H), 2.6-2.4 (m, 4H), 1.9-1.4 (m, 8H), 1.0 (t, 3H) ppm. HPLC analysis (5:10:85 H₂O:A1:MeOH) showed a purity of 95% with a retention time of 2.7 min.

### N-(2-{[2-(2,6-Dimethylphenoxy)ethyl]amino}-5-propoxyphenyl)-2-piperidylacetamide (45)

A 100 mL 3-neck flask fitted with a stir-bar, condenser, and an Ar inlet was charged with aniline 44 (3.00 g, 10.3 mmol), bromide 31 (3.55 g, 15.5 mmol), K₂CO₃ (2.56 g, 18.5 mmol), KI (166 mg, 1.00 mmol), and DMF (30 mL). The suspension was heated in a 120° C. bath overnight. Another 1 g of bromide 31 and 50 mg of KI were added, and the mixture was heated for another 2 hr to completion as monitored by HPLC. The suspension was then cooled. The solids were filtered off and rinsed with EtOAc (75 mL). The filtrate was washed with saturated aqueous NH₄Cl (2×50 mL), H₂O (2×50 mL), and brine (50 mL), filtered through phase separation paper, and concentrated in vacuo to give 6.3 g of a brown solid. The crude material was chromatographed on silica gel eluting the product with 10-15% EtOAc in hexanes and giving material of −50% purity. The combined product fractions were concentrated in vacuo to 300 mL and extracted with 5% aqueous citric acid (2×125 mL). The combined extracts were brought to a pH of 5.5 with saturated NaHCO₃ (250 mL) and extracted with EtOAc (125 mL). The organic phase was washed with saturated NaHCO₃ (50 mL) and brine (50 mL), filtered through phase separation paper, and concentrated in vacuo to give 1.2 g of a yellow solid. The solid was collected with hexanes (10 mL) and dried to give 750 mg of 45 as a white crystalline solid (17%). ¹H NMR (300 MHz, CDCl₃): δ 9.46 (bs, 1H), 7.45 (d, 1H), 7.06-6.92 (m, 3H), 6.86 (d, 1H), 6.70 (dd, 1H), 4.02 (t, 2H), 3.92 (t, 2H), 3.92 (bs, 1H), 3.42 (qt, 2H), 3.13 (s, 2H), 2.56 (bm, 4H), 2.32 (s, 6H), 1.80 (sept, 2H), 1.63 (m, 4H), 1.45 (m, 2H), 1.05 (t, 3H) ppm. HPLC analysis (0:10:90 H₂O:A1:MeOH) showed a purity of greater than 99% with a retention time of 3.6 min.

### 1-[2-(2,6-Dimethylphenoxy)-ethyl]-2-piperidin-1-ylmethyl-5-propoxy-1H-benzimidazole (Compound #5). A 50 mL flask fitted with a stir-bar, condenser, and an Ar inlet was charged with amine 45 (730 mg, 1.66 mmol) and AcOH (10 mL). The resultant solution was heated in a 100° C. bath for 3 hr. The mixture was cooled and concentrated to an orange oil. The oil was taken up in EtOAc (25 mL) and washed with saturated NaHCO₃ (3×25 mL) and brine (25 mL). The solution was filtered through phase separation paper and concentrated to an orange oil, which crystallized overnight. The crystals were filtered with ice-cold hexanes (5 mL) and rinsed with hexanes (2×1 mL) to give 410 mg of an off-white solid. A second crop of 40 mg was filtered from the mother liquors. The combined material yielded 450 mg of Compound #5 as an off-white solid (84%). ¹H NMR (300 MHz, CDCl₃): δ 7.35 (d, 1H), 7.24 (d, 1H), 7.00-6.86 (m, 4H), 4.76 (t, 2H), 4.14 (t, 2H), 3.99 (t, 2H), 3.85 (bs, 2H), 2.46 (bm, 4H), 2.02 (s, 6H), 1.85 (sept, 2H), 1.60-1.40 (m, 6H), 1.07 (t, 3H) ppm. HPLC analysis (0:10:90 H₂O:A1:MeOH) showed a purity of greater than 99% with retention time of 4.5 min.

Any compound of the invention whose synthesis is not explicitly exemplified herein can either be prepared by following the procedure of structurally similar compounds that have been exemplified herein, or is available from commercial vendors or companies that sell compounds for screening. For example, compound #1 (RN: 931337-74-5), whose synthesis has been shown above is commercially available from Aurora Fine Chemicals. Similarly, the compound of formula

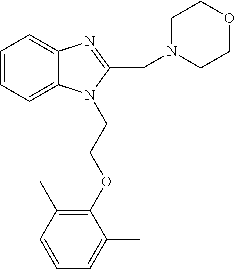 [SMILES: Cc1cccc(C)c1OCCn1c(CN2CCOCC2)nc2ccccc21]

(RN 92480-63-5) is available from Ambinter.

### Assays and Results:

To identify compounds that block persistent sodium currents, they were tested in a sodium depletion/repletion assay in which changes in the fluorescence of voltage-sensitive dyes measured in a Fluorometric Imaging Plate Reader (FLIPR) were used to infer the effects of the compounds on persistent sodium currents. The compounds were first tested in a single concentration HTS protocol. Those compounds that produced more than 80% inhibition of the control response were further tested in a 8-point concentration-response format, and the compound's antagonism of persistent current is expressed as its IC₅₀ (see Table 1 below).

To determine the selectivity of effects of these compounds on the persistent current, the inhibition of transient sodium currents using an automated patch clamp was also tested for the compounds. For direct measurement of Na+ currents the IonWorks automated patch clamp system was used. The IonWorks system is a high-throughput electrophysiological instrument that performs whole-cell voltage clamp recordings in a 384-well plate format.

Despite their utility in screening and identifying compounds, the FLIPR and IonWorks assays possess some disadvantages. The FLIPR assay cannot regulate cellular membrane potential and does not directly measure current conductance of VGSCs. In contrast, the IonWorks assay addresses these shortcomings, but does not produce the necessary gigohm seals to evaluate persistent current. Therefore, the efficacy and selectivity of the identified persistent current antagonists were verified using conventional gigohm-seal manual patch-clamp (MPC).

MPC experiments were conducted on human embroynic kidney (HEK) cells stably transfected with sodium channels were plated on coverslips. Cells were patch clamped using standard patch-clamp techniques (Hamill, O. P., A. Marty, et al., 1981 Pflugers Arch. 391:85-100). Once sufficient persistent current was established, buffer was introduced into the chamber at a constant flow rate of 0.5-1 minute/mL until the persistent current stabilized. Subsequent concentration-response experiments were conducted using this same flow rate. After application of the compound, current amplitude was monitored until equilibrium was established. The data recorded at equilibrium was normalized to the control current measured during perfusion with control buffer in the absence of a test compound. Adequate solution perfusion and gigohm seal stability were monitored throughout the experiment. The compound's antagonism of persistent or transient sodium currents is expressed as the IC5O of block for the respective current (see Table 1). Selectivity for block of the persistent current was calculated as the ratio of the IC5O for the transient current divided by the IC5O for the persistent current.

Direct measurement of the sodium current in cells expressing either Naᵥ1.3 or

NaᵥI.6 channels using gigohm seal MPC clearly demonstrated a strong selectivity of block. As shown for the exemplary compounds (compound #1 in Table 1 and Compound A shown below)

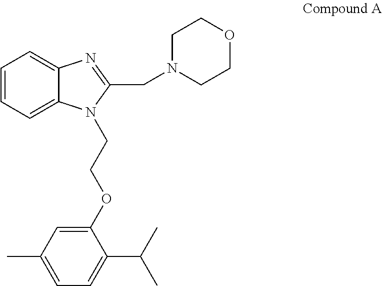 [SMILES: Cc1ccc(C(C)C)c(OCCn2c(CN3CCOCC3)nc3ccccc32)c1]

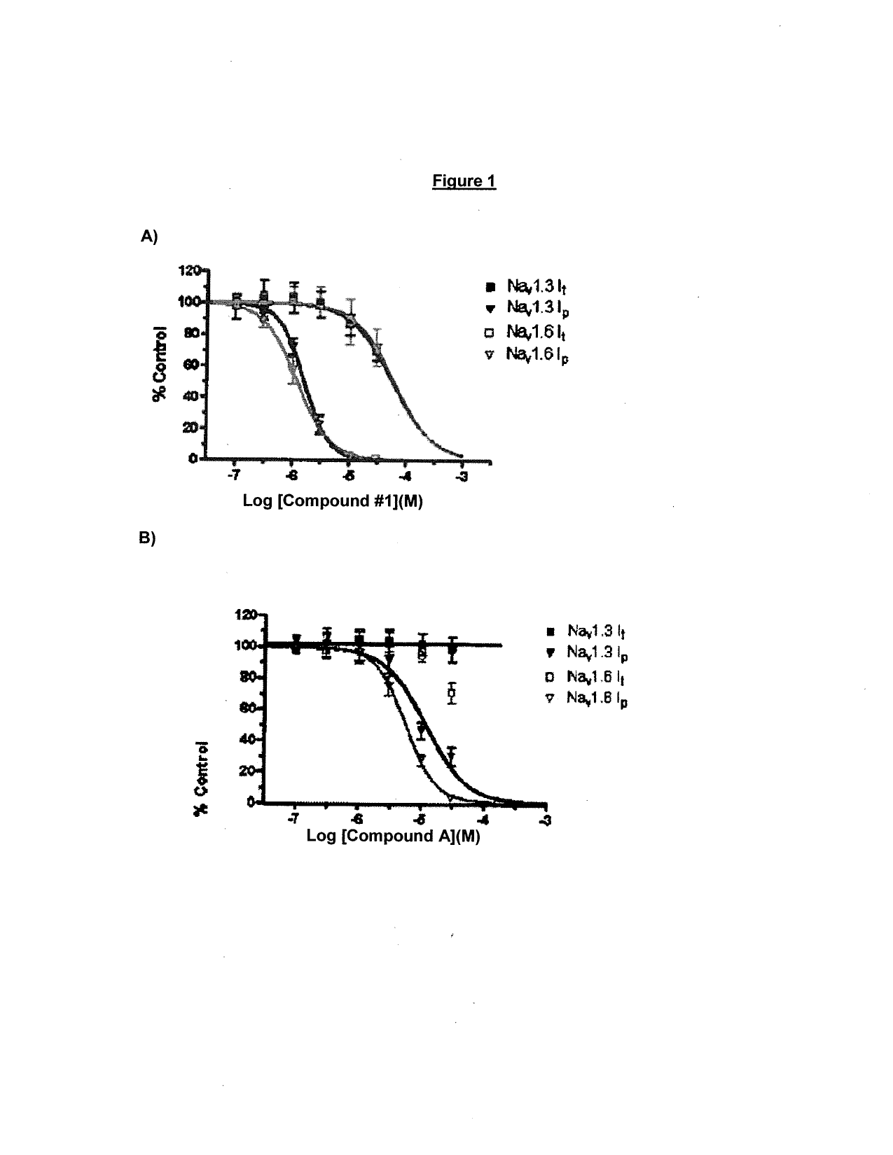

in FIG. 1, low to submicromolar concentrations of these compounds were effective in blocking 50% of the persistent current while the effective dose for blocking the transient current greatly exceeded 30 μM.

Activity of the compounds on the rate of spontaneous firing of neurons was assessed in hippocampal neurons. Hippocampal cells from P-2 rats were cultured for 10-14 days prior to use. Cells were plated on laminin-coated cover slips that could be transferred to a perfusion chamber for conventional whole cell electrophysiological studies. Conventional whole cell current clamp methods were used to record steady state and spontaneous action potentials. Cells were held at their resting potential (Iclamp=0 pA) and spontaneous action potentials were recorded and followed over time. Healthy cells had low rates of spontaneous firing. Spontaneous firing rate increased with metabolic insults to cells, e.g.Mg²⁺-depletion or with aging. Spontaneous firing appeared to occur more often in older cell cultures (10-14 days).

### In Vivo Pain Assays: Reduction of Mechanical Allodynia and Thermal Hyperalgesia

To assess the actions of the presently claimed benzimdazole compounds in neuropathic pain their efficacy in reversing the mechanical allodynia produced by spinal nerve ligation (Chung Model) and reducing the thermal hyperalgesia produced by subcutaneous injection of capsaicin was determined.

In the Chung spinal nerve ligation model, rats are anesthetized before surgery. The surgical site is shaved and prepared either with betadine or Novacaine. Incision is made from the thoracic vertebra XIII down toward the sacrum. Muscle tissue is separated from the spinal vertebra (left side) at the L4-S2 levels. The L6 vertebra is located and the transverse process is carefully removed with a small rongeur to expose the L4-L6 spinal nerves. The L5 and L6 spinal nerves are isolated and tightly ligated with 6-0 silk thread. The same procedure is done on the right side as a control, except no ligation of the spinal nerves is performed.

A complete hemostasis is confirmed, then the wounds are sutured. A small amount of antibiotic ointment is applied to the incised area, and the rat is transferred to the recovery plastic cage under a regulated heat temperature lamp.

Rats recovering from the surgery gain weight and display a level of general activity similar to that of normal rats. However, these rats develop abnormalities of the foot, wherein the hindpaw is moderately everted and the toes are held together. More importantly, the hindpaw on the side affected by the surgery appears to become sensitive to pain from low-threshold mechanical stimuli, such as that producing a faint sensation of touch in a human, within about 1 week following surgery. This sensitivity to normally non-painful touch is called “tactile allodynia” and lasts for at least two months. The response includes lifting the affected hindpaw to escape from the stimulus, licking the paw and holding it in the air for many seconds. None of these responses is normally seen in the control group.

Compounds to be tested are prepared as suspension in vehicle containing 2% sodium carboxymethylcellulose (CMC) and 1% Pluronic Fl 27 by vigorous homogenization. On the day of the experiment, at least seven days after the surgery, six rats per test group are administered the test compounds/drugs or vehicle by intraperitoneal (i.p.) injection. Tactile allodynia is measured prior to and at increments of 15, 30, 60 and 90 minutes after drug administration using von Frey hairs that are a series of fine hairs with incremental differences in stiffness. Rats are placed in a plastic cage with a wire mesh bottom and allowed to acclimate for approximately 30 minutes. The von Frey hairs are applied perpendicularly through the mesh to the mid-plantar region of the rats' hindpaw with sufficient force to cause slight buckling and held for 6-8 seconds. The applied force is calculated to range from 0.41 to 15.1 grams. If the paw is sharply withdrawn, it is considered a positive response. A normal animal will not respond to stimuli in this range, but a surgically ligated paw will be withdrawn in response to a 1-2 gram hair.

A second method for evaluating of 1,2 substituted benzimidazoles on neuropathic pain is to determine their effects on capsaicin-induced thermal hyperalgesia in the rat.

Peripherally administered capsaicin (the active agent in chili peppers) induces an acute, local, inflammatory response through actions on nociceptive sensory nerve endings (“pain fibers”). In rats, intraplantar injection of capsaicin produces decease in withdrawal latency to radiant heat (thermal hyperalgesia). This primary hyperalgesia, observed at the site of injury, is characterized by sensitization to thermal and mechanical stimulation. Primary hyperalgesia, especially that elicited by noxious thermal stimulus, is mediated, in part, by sensitization of C-fiber mechanoheat (polymodal) receptors (Kennis, 1982; Konietzny and Hensel, 1983; Simone, et al, 1987). This rat model has been used to identify small molecule therapeutics, including VR-1

and CB1 receptor antagonists. The purpose of this study is to determine the pharmacological efficacy of the present compounds in this model of thermal hyperalgesia.

Intraplantar injection of capsaicin (30 pg) is used to induce thermal hyperalgesia, as described previously (Gilchrist et al., 1996). The plantar test is used to assess capsaicin-induced thermal hyperalgesia (n=6/group). For this test, hind paw withdrawal latencies (PWLs) to a noxious thermal stimulus are determined using the technique described by Hargreaves et al. (1988) using a plantar test apparatus (PAW

Thermal Analgesia Meter instrument Department of Anesthesiology, UCSD, San Diego, Calif.). Time to remove paw from heat source is measured and expressed as the paw withdrawal latency. Cut-off is set at 20.48 seconds, and any directed paw withdrawal from the heat source is taken as the endpoint. The glass plate temperature is set at 25° C. and the light intensity at 4.8 Amperes.

Compounds to be tested are prepared as suspension in vehicle containing 2% sodium carboxymethylcellulose (CMC) and 1% Pluronic Fl 27 by vigorous homogenization. On the day of the experiment, six rats per test group are administered the test drugs or vehicle by intraperitoneal (i.p.) injection 30 minutes prior to intraplantar injection of capsaicin. Post-capsaicin paw withdrawal latency are assessed 30 nm utes after capsaicin challenge. Pre-capsaicin and post-capsaicin values arecompared using a repeated measures one-way ANOVA and pairwise Multiple Comparison Procedures (Dunns method).

TABLE 1

| | FLIPR IC50 | | |
|---|---|---|---|
| Structure | (μM) | IW IC50 (μM) | HTS % Block |
| 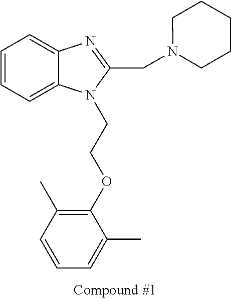 [SMILES: Cc1cccc(C)c1OCCn1c(CN2CCCCC2)nc2ccccc21] | 0.066 | >30.00 | |
| 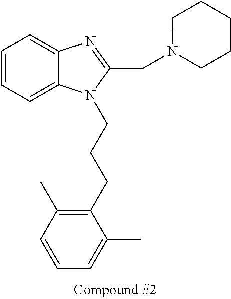 [SMILES: Cc1cccc(C)c1CCCn1c(CN2CCCCC2)nc2ccccc21] | | | |
| 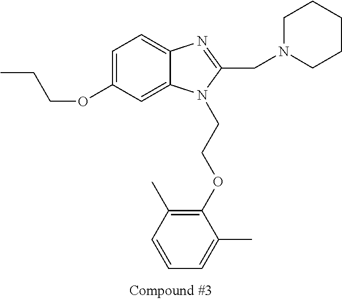 [SMILES: CCCOc1ccc2nc(CN3CCCCC3)n(CCOc3c(C)cccc3C)c2c1] | | | |
| 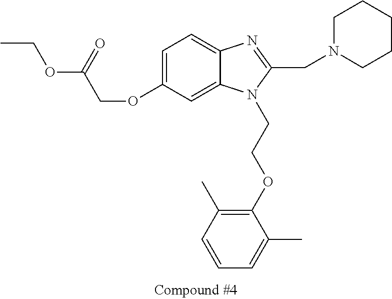 [SMILES: CCOC(=O)COc1ccc2nc(CN3CCCCC3)n(CCOc3c(C)cccc3C)c2c1] | | | |
| 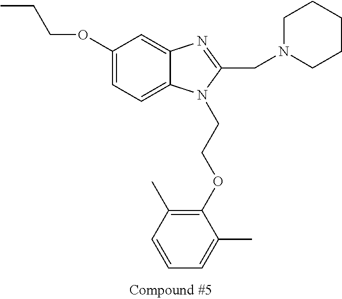 [SMILES: CCCOc1ccc2c(c1)nc(CN1CCCCC1)n2CCOc1c(C)cccc1C] | | | |
| 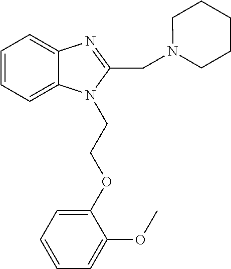 [SMILES: COc1ccccc1OCCn1c(CN2CCCCC2)nc2ccccc21] | 0.347 | >30.00 | 94.8 |
| 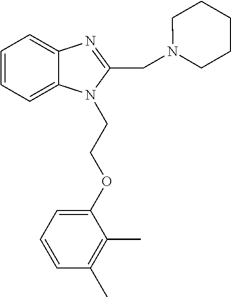 [SMILES: Cc1cccc(OCCn2c(CN3CCCCC3)nc3ccccc32)c1C] | 0.452 | 13.77 | 78.9 |
| 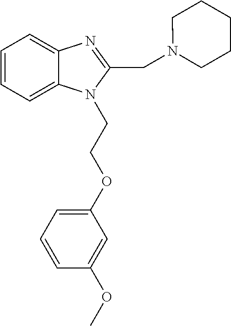 [SMILES: COc1cccc(OCCn2c(CN3CCCCC3)nc3ccccc32)c1] | 0.521 | >30.00 | 96.5 |
| 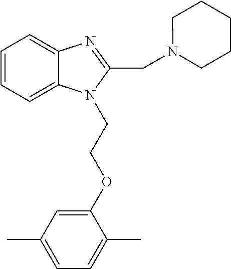 [SMILES: Cc1ccc(C)c(OCCn2c(CN3CCCCC3)nc3ccccc32)c1] | 0.559 | 22.75 | 88.9 |
| 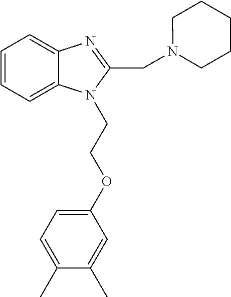 [SMILES: Cc1ccc(OCCn2c(CN3CCCCC3)nc3ccccc32)cc1C] | 0.589 | 23.44 | 80.1 |
| 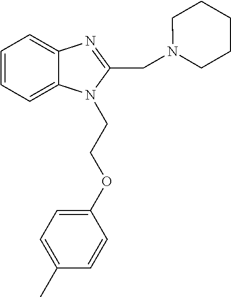 [SMILES: Cc1ccc(OCCn2c(CN3CCCCC3)nc3ccccc32)cc1] | 0.751 | >30.00 | 94.6 |
| 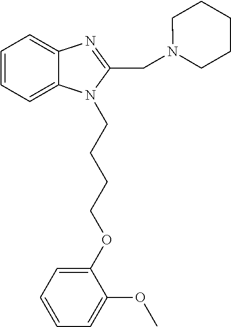 [SMILES: COc1ccccc1OCCCCn1c(CN2CCCCC2)nc2ccccc21] | 0.830 | >30.00 | 95.5 |
| 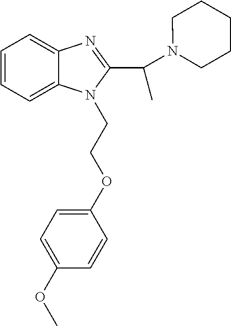 [SMILES: COc1ccc(OCCn2c(C(C)N3CCCCC3)nc3ccccc32)cc1] | 0.950 | >30.00 | 95.2 |
| 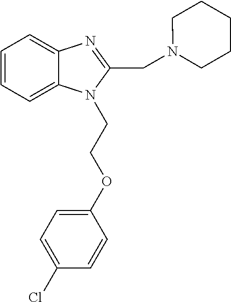 [SMILES: Clc1ccc(OCCn2c(CN3CCCCC3)nc3ccccc32)cc1] | 1.462 | 27.75 | 80.7 |
| 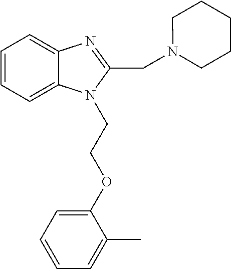 [SMILES: Cc1ccccc1OCCn1c(CN2CCCCC2)nc2ccccc21] | 5.561 | >30.00 | 93.6 |
| 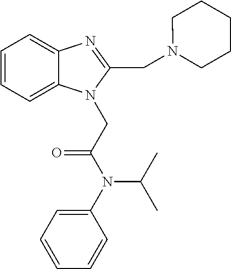 [SMILES: CC(C)N(C(=O)Cn1c(CN2CCCCC2)nc2ccccc21)c1ccccc1] | 6.348 | >30.00 | 84.2 |
| 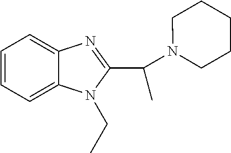 [SMILES: CCn1c(C(C)N2CCCCC2)nc2ccccc21] | 16.251 | >30.00 | 90.6 |
| 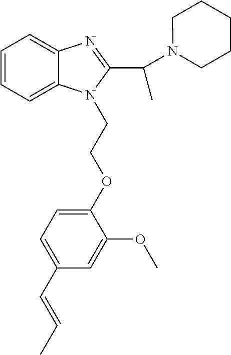 [SMILES: C/C=C/c1ccc(OCCn2c(C(C)N3CCCCC3)nc3ccccc32)c(OC)c1] | >30.000 | >30.00 | 34.6 |
| 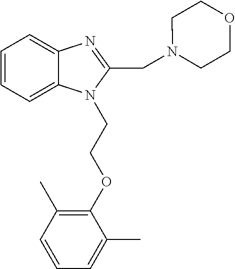 [SMILES: Cc1cccc(C)c1OCCn1c(CN2CCOCC2)nc2ccccc21] | 0.729 | >30.00 | 95.2 |
| 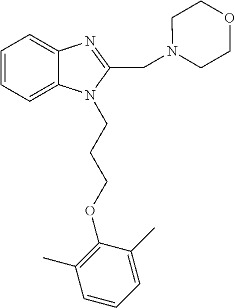 [SMILES: Cc1cccc(C)c1OCCCn1c(CN2CCOCC2)nc2ccccc21] | 0.852 | >30.00 | 98.9 |
| 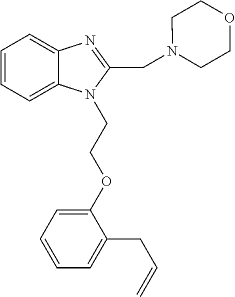 [SMILES: C=CCc1ccccc1OCCn1c(CN2CCOCC2)nc2ccccc21] | 1.136 | >30.00 | 91.5 |
| 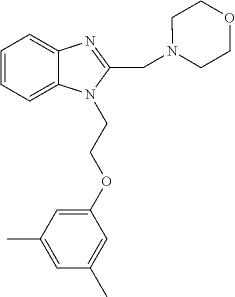 [SMILES: Cc1cc(C)cc(OCCn2c(CN3CCOCC3)nc3ccccc32)c1] | 1.465 | >30.00 | 92.6 |
| 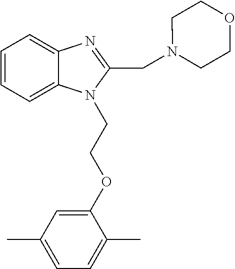 [SMILES: Cc1ccc(C)c(OCCn2c(CN3CCOCC3)nc3ccccc32)c1] | 2.300 | >30.00 | 92.7 |
| 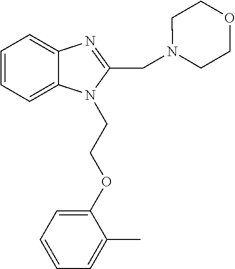 [SMILES: Cc1ccccc1OCCn1c(CN2CCOCC2)nc2ccccc21] | 3.243 | >30.00 | 92.7 |
| 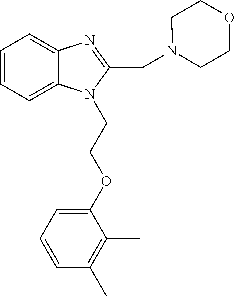 [SMILES: Cc1cccc(OCCn2c(CN3CCOCC3)nc3ccccc32)c1C] | 3.533 | >30.00 | 97.3 |
| 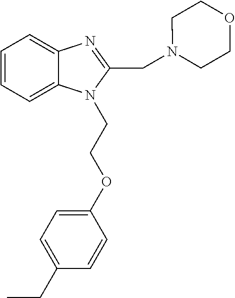 [SMILES: CCc1ccc(OCCn2c(CN3CCOCC3)nc3ccccc32)cc1] | 3.666 | 29.99 | 90.8 |
| 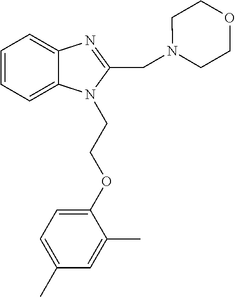 [SMILES: Cc1ccc(OCCn2c(CN3CCOCC3)nc3ccccc32)c(C)c1] | 4.518 | >30.00 | 79.3 |
| 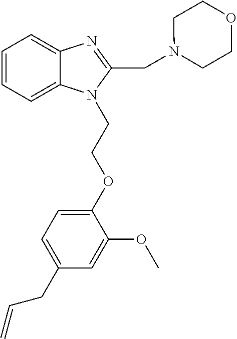 [SMILES: C=CCc1ccc(OCCn2c(CN3CCOCC3)nc3ccccc32)c(OC)c1] | 5.122 | 30.00 | 70.7 |
| 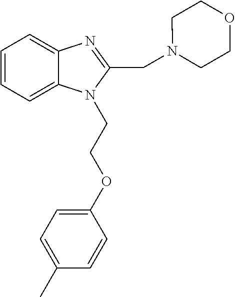 [SMILES: Cc1ccc(OCCn2c(CN3CCOCC3)nc3ccccc32)cc1] | 6.380 | >30.00 | 92.3 |
| 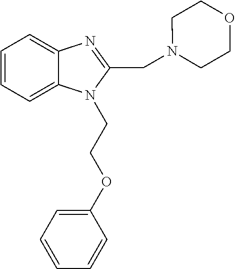 [SMILES: c1ccc(OCCn2c(CN3CCOCC3)nc3ccccc32)cc1] | 7.793 | >30.00 | 99.2 |
| 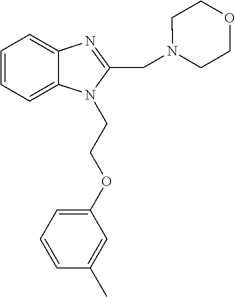 [SMILES: Cc1cccc(OCCn2c(CN3CCOCC3)nc3ccccc32)c1] | 7.848 | >30.00 | 86.5 |
| 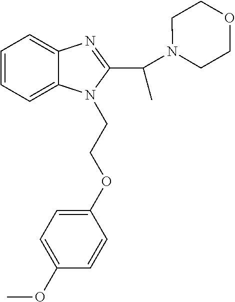 [SMILES: COc1ccc(OCCn2c(C(C)N3CCOCC3)nc3ccccc32)cc1] | 9.810 | >30.00 | 92.9 |
| 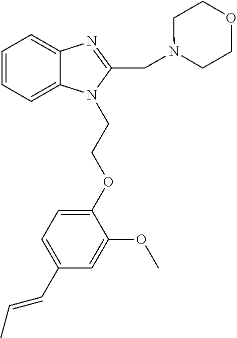 [SMILES: C/C=C/c1ccc(OCCn2c(CN3CCOCC3)nc3ccccc32)c(OC)c1] | 18.230 | >30.00 | 75.7 |
| 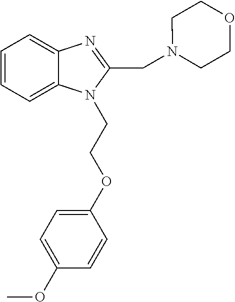 [SMILES: COc1ccc(OCCn2c(CN3CCOCC3)nc3ccccc32)cc1] | >30.00 | >30.00 | 57.7 |
| 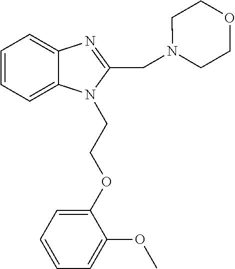 [SMILES: COc1ccccc1OCCn1c(CN2CCOCC2)nc2ccccc21] | >30.00 | >30.00 | 70.1 |

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered orally, subcutaneously, intravenously, intrathecally or some suitable combination(s) thereof.

In addition to the common dosage forms set out above, the compounds of this invention may also be administered by controlled release means and/or delivery devices such as those described in U.S. Pat. Nos. 3,845,770; 3,916,899; 3,536,809; 3,598,123; 3,630,200; 4,008,719; and 5,366,738 the disclosures of which are incorporated herein by reference.

For use where a composition for intravenous administration is employed, a suitable daily dosage range for anti-inflammatory, anti-atherosclerotic or anti-allergic use is from about 0.001 mg to about 25 mg (preferably from 0.01 mg to about 1 mg) of a compound of this invention per kg of body weight per day and for cytoprotective use from about 0.1 mg to about 100 mg (preferably from about 1 mg to about 100 mg and more preferably from about 1 mg to about 10 mg) of a compound of this invention per kg of body weight per day. For the treatment of diseases of the eye, ophthalmic preparations for ocular administration comprising 0.001-1% by weight solutions or suspensions of the compounds of this invention in an acceptable ophthalmic formulation may be used.

Preferably, the pharmaceutical preparation is in a unit dosage form. In such form, the preparation is subdivided into suitably sized unit doses containing appropriate quantities of the active component, e.g., an effective amount to achieve the desired purpose.

The magnitude of prophylactic or therapeutic dose of a compound of this invention will, of course, vary with the nature of the severity of the condition to be treated and with the particular compound and its route of administration. It will also vary according to the age, weight and response of the individual patient. It is understood that a specific daily dosage amount can simultaneously be both a therapeutically effective amount, e.g., for treatment to slow progression of an existing condition, and a prophylactically effective amount, e.g., for prevention of condition.

The quantity of active compound in a unit dose of preparation may be varied or adjusted from about 0.001 mg to about 500 mg. In one embodiment, the quantity of active compound in a unit dose of preparation is from about 0.01 mg to about 250 mg. In another embodiment, the quantity of active compound in a unit dose of preparation is from about 0.1 mg to about 100 mg. In another embodiment, the quantity of active compound in a unit dose of preparation is from about 1.0 mg to about 100 mg. In another embodiment, the quantity of active compound in a unit dose of preparation is from about 1.0 mg to about 50 mg. In still another embodiment, the quantity of active compound in a unit dose of preparation is from about 1.0 mg to about 25 mg.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.01 mg/day to about 2000 mg/day of the compounds of the present invention. In one embodiment, a daily dosage regimen for oral administration is from about 1 mg/day to 1000 mg/day. In another embodiment, a daily dosage regimen for oral administration is from about 1 mg/day to 500 mg/day. In another embodiment, a daily dosage regimen for oral administration is from about 100 mg/day to 500 mg/day. In another embodiment, a daily dosage regimen for oral administration is from about 1 mg/day to 250 mg/day. In another embodiment, a daily dosage regimen for oral administration is from about 100 mg/day to 250 mg/day. In still another embodiment, a daily dosage regimen for oral administration is from about 1 mg/day to 100 mg/day. In still another embodiment, a daily dosage regimen for oral administration is from about 50 mg/day to 100 mg/day. In a further embodiment, a daily dosage regimen for oral administration is from about 1 mg/day to 50 mg/day. In another embodiment, a daily dosage regimen for oral administration is from about 25 mg/day to 50 mg/day. In a further embodiment, a daily dosage regimen for oral administration is from about 1 mg/day to 25 mg/day. The daily dosage may be administered in a single dosage or can be divided into from two to four divided doses.

In one aspect, the present invention provides a kit comprising a therapeutically effective amount of at least one compound of the present invention, or a pharmaceutically acceptable salt of said compound and a pharmaceutically acceptable carrier, vehicle or diluents, and directions for the use of said kit.

Each and every reference disclosed in the specification, whether non-patent (e.g., scientific, journal references) or patent (e.g., granted patents or published patent

applications) is incorporated herein by reference in its entirety for all purposes.

The foregoing descriptions details specific methods and compositions that can be

employed to practice the present invention, and represents the best mode comtemplated. It should not be construed as limiting the overall scope hereof; rather, the ambit of the present invention is to be governed only by the lawful construction of the appended claims.

What is claimed is:

1. A compound selected from the group consisting of: 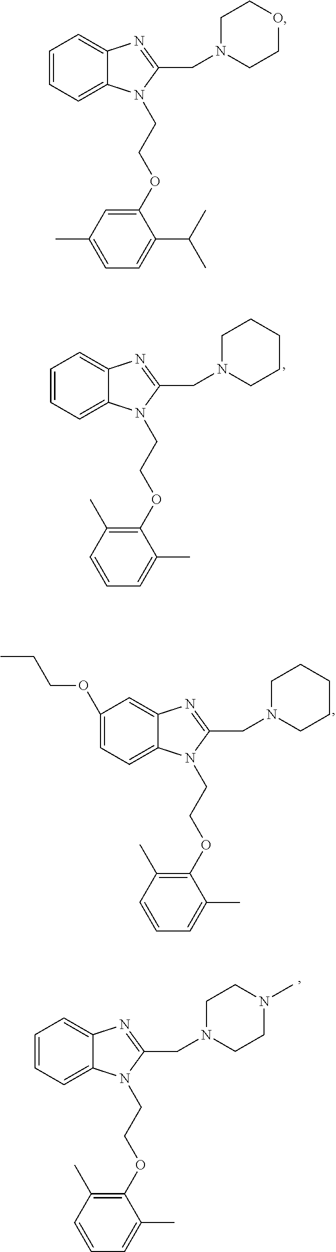 [SMILES: Cc1ccc(C(C)C)c(OCCn2c(CN3CCOCC3)nc3ccccc32)c1 ; Cc1cccc(C)c1OCCn1c(CN2CCCCC2)nc2ccccc21 ; CCCOc1ccc2c(c1)nc(CN1CCCCC1)n2CCOc1c(C)cccc1C ; Cc1cccc(C)c1OCCn1c(CN2CCN(C)CC2)nc2ccccc21] 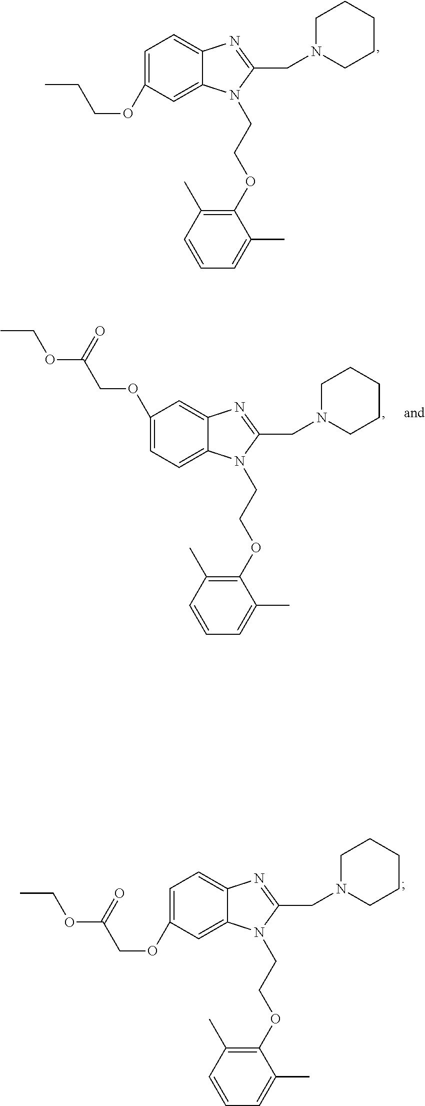 [SMILES: CCCOc1ccc2nc(CN3CCCCC3)n(CCOc3c(C)cccc3C)c2c1 ; CCOC(=O)COc1ccc2c(c1)nc(CN1CCCCC1)n2CCOc1c(C)cccc1C ; CCOC(=O)COc1ccc2nc(CN3CCCCC3)n(CCOc3c(C)cccc3C)c2c1]
or a pharmaceutically acceptable salt thereof.

2. A pharmaceutical composition comprising at least one compound of claim 1, or a pharmaceutically acceptable salt thereof, and at least one pharmaceutically acceptable carrier.

3. A method of blocking persistent sodium current without affecting transient current in a mammal comprising administering to said mammal in need thereof a therapeutically effective amount of the compound of Formula I 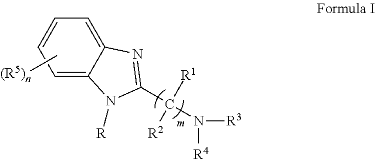
or a pharmaceutically acceptable salt thereof; wherein:
R is C₁₋₆ alkyl, which is unsubstituted or substituted with a substituent selected from the group consisting of —N(C₁₋₆ alkyl)₂, C₆₋₁₂ aryl, C₆₋₁₂ aryloxy, heteroaryl, and —C(═O)—N(R⁶)—C₆₋₁₂ aryl;
each R¹ and R² are independently H or C₁₋₆ alkyl;
R³ and R⁴ together with the nitrogen atoms to which they are shown attached form a five- or six-membered heterocyclyl group selected from the group consisting of pyrrolidinyl, piperidinyl, piperazinyl, and morpholinyl, each of which is independently unsubstituted or substituted with a ring system substituent;
R⁵ is selected from the group consisting of C₁₋₆ alkyl, and C₁₋₆ alkoxy, wherein said C₁₋₆ alkyl and the “alkyl” portion of said C₁₋₆ alkoxy are independently unsubstituted or substituted with a substituent selected from the group consisting of hydroxyl, —C(═O)OH, —C(═O)O—C₁₋₆ alkyl, —C(═O)-heterocyclyl, and —N(C₁₋₆ alkyl)₂;
R⁶ is H or C₁₋₆ alkyl;
m is 1 or 2; and
n is 0 or 1.

4. A method of treating a disease or condition in a mammal, wherein said disease or condition is mediated by elevated persistent sodium current, comprising administering to said mammal in need thereof an effective amount of a compound of Formula I 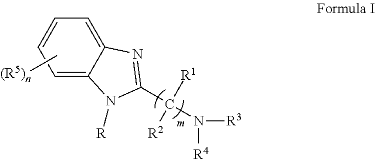
or a pharmaceutically acceptable salt thereof; wherein:
R is C₁₋₆ alkyl, which is unsubstituted or substituted with a substituent selected from the group consisting of —N(C₁₋₆ alkyl)₂, C₆₋₁₂ aryl, C₆₋₁₂ aryloxy, heteroaryl, and —C(═O)—N(R⁶)—C₆₋₁₂ aryl;
each R¹ and R² are independently H or C₁₋₆ alkyl;
R³ and R⁴ together with the nitrogen atoms to which they are shown attached form a five- or six-membered heterocyclyl group selected from the group consisting of pyrrolidinyl, piperidinyl, piperazinyl, and morpholinyl, each of which is independently unsubstituted or substituted with a ring system substituent;
R⁵ is selected from the group consisting of C₁₋₆ alkyl, and C₁₋₆ alkoxy, wherein said C₁₋₆ alkyl and the “alkyl” portion of said C₁₋₆ alkoxy are independently unsubstituted or substituted with a substituent selected from the group consisting of hydroxyl, —C(═O)OH, —C(═O)O—C₁₋₆ alkyl, —C(═O)-heterocyclyl, and —N(C₁₋₆ alkyl)₂;
R⁶ is H or C₁₋₆ alkyl;
m is 1 or 2; and
n is 0 or 1.

5. The method of claim 4, wherein said disease or condition is selected from the group consisting of chronic pain, ocular disorder, multiple sclerosis, and seizure disorder.

6. The method of claim 5, wherein the ocular disorder is selected from the group consisting of age related macular degeneration (AMD), geographic atrophy (GA), retinitis pigmentosa, Stargardt's disease cone dystrophy, and pattern dystrophy of the retinal pigmented epithelium, macular edema, retinal detachment, retinal trauma, retinal tumors and retinal diseases associated with said tumors, congenital hypertrophy of the retinal pigmented epithelium, acute posterior multifocal placoid pigment epitheliopathy, optic neuritis, acute retinal pigment epithelitis, optic neuropathies and glaucoma; the chronic pain is selected from the group consisting of neuropathic pain, inflammatory pain, visceral pain, post-operative pain, pain resulting from cancer or cancer treatment, headache pain, irritable bowel syndrome pain, fibromyalgia pain, and pain resulting from diabetic neuropathy; and the seizure disorder is selected from the group consisting of epilepsy and chemically-induced seizure disorder.

7. The method of claim 4, wherein R is C₁₋₆ alkyl, which is unsubstituted or substituted with a substituent selected from the group consisting of C₆₋₁₂ aryloxy and —C(═O)—N(R⁶)—C₆₋₁₂ aryl.

8. The method of claim 7, wherein the “aryl” portion of said C₆₋₁₂ aryloxy and —C(═O)—N(R⁶)—C₆₋₁₂ aryl groups is independent unsubstituted or substituted with 1-3 substituents independently selected from the group consisting of halo, C₁₋₆ alkyl, C₂₋₆ alkenyl, and C₁₋₆ alkoxy, wherein the C₁₋₆ alkyl or the “alkyl” portion of said C₁₋₆ alkoxy is independently unsubstituted or substituted with an alkenyl substituent.

9. The method of claim 7, wherein R is C₁₋₆ alkyl which is substituted with a C₆₋₁₂ aryloxy substitutent, and wherein the “aryl” portion of said C₆₋₁₂ aryloxy substitutent is unsubstituted or substituted with one to two substituents independently selected from the group consisting of chlro, methyl, isopropyl, methoxy, 1-propenyl, and 2-propenyl.

10. The method of claim 7, wherein R is selected from the group consisting of: Ethyl,

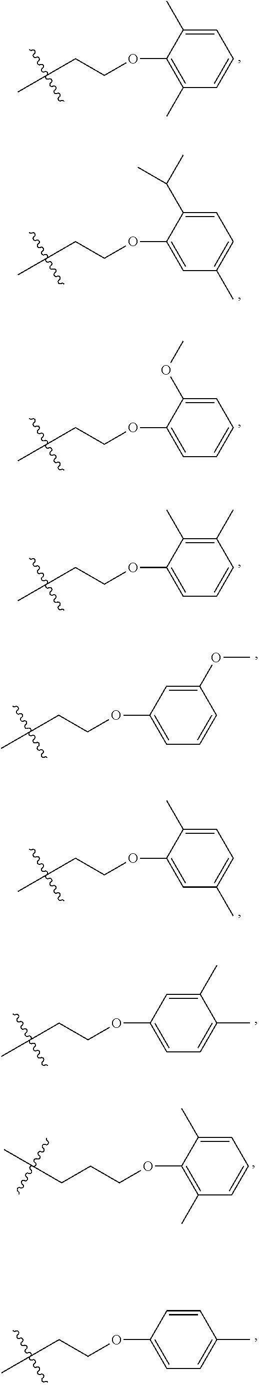

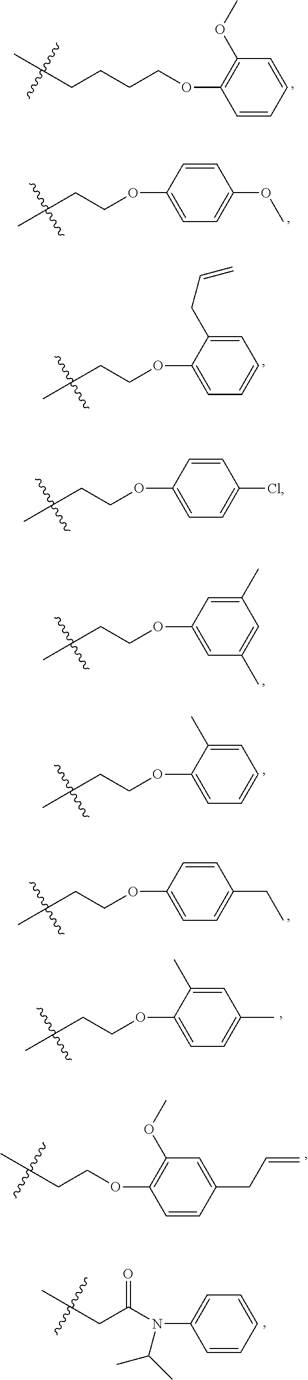

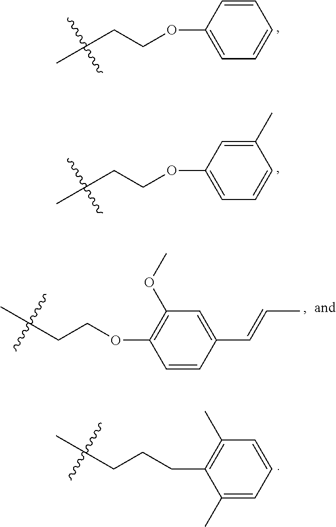

11. The method of claim 4, wherein R¹ and R² are both H.

12. The method of claim 4, wherein one of R¹ and R² is C₁₋₆ alkyl, and the other is H.

13. The method of claim 4, wherein one of R¹ and R² is methyl, and the other is H.

14. The method of claim 4, wherein m is 1.

15. The method of claim 4, wherein R³ and R⁴ together with the nitrogen atoms to which they are shown attached form a six-membered heterocyclyl group selected from the group consisting of piperidinyl, and morpholinyl, each of which is independently unsubstituted or substituted with a ring system substituent.

16. The method of claim 15, wherein each of said piperidinyl and morpholinyl is independently unsubstituted or substituted with a C₁₋₆ alkyl.

17. The method of claim 4, wherein n is 0.

18. The method claim 4, wherein n is 1.

19. The method of claim 4, wherein R⁵ is C₁₋₆ alkoxy, wherein the “alkyl” portion of said C₁₋₆ alkoxy is unsubstituted or substituted with a —C(═O)O—C₁₋₆ alkyl.

20. The method of claim 19, wherein R⁵ is selected from the group consisting of —O—CH₂CH₂CH₃ and —O—CH₂—C(═O)—O—CH₂CH₃.

21. The method of claim 4, wherein the compound of Formula I is selected from the group consisting of: 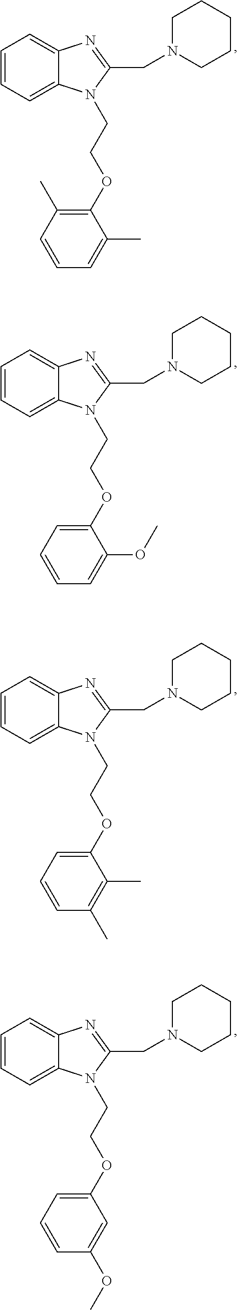 [SMILES: Cc1cccc(C)c1OCCn1c(CN2CCCCC2)nc2ccccc21 ; COc1ccccc1OCCn1c(CN2CCCCC2)nc2ccccc21 ; Cc1cccc(OCCn2c(CN3CCCCC3)nc3ccccc32)c1C ; COc1cccc(OCCn2c(CN3CCCCC3)nc3ccccc32)c1] 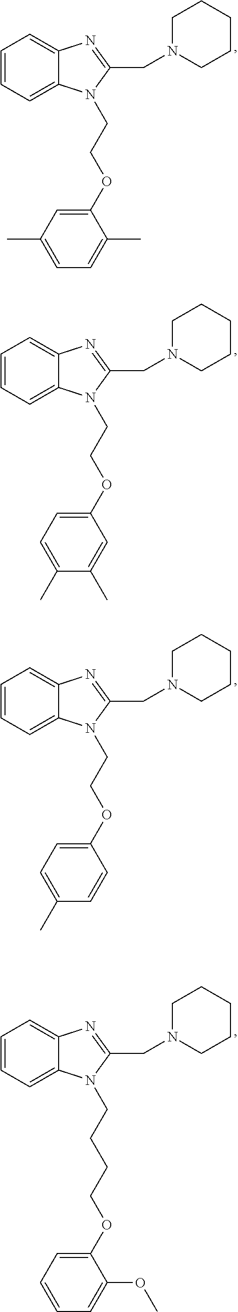 [SMILES: Cc1ccc(C)c(OCCn2c(CN3CCCCC3)nc3ccccc32)c1 ; Cc1ccc(OCCn2c(CN3CCCCC3)nc3ccccc32)cc1C ; Cc1ccc(OCCn2c(CN3CCCCC3)nc3ccccc32)cc1 ; COc1ccccc1OCCCCn1c(CN2CCCCC2)nc2ccccc21] 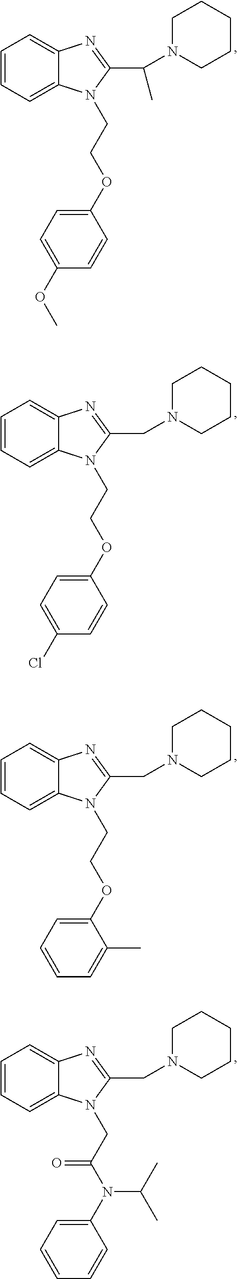 [SMILES: COc1ccc(OCCn2c(C(C)N3CCCCC3)nc3ccccc32)cc1 ; Clc1ccc(OCCn2c(CN3CCCCC3)nc3ccccc32)cc1 ; Cc1ccccc1OCCn1c(CN2CCCCC2)nc2ccccc21 ; CC(C)N(C(=O)Cn1c(CN2CCCCC2)nc2ccccc21)c1ccccc1] 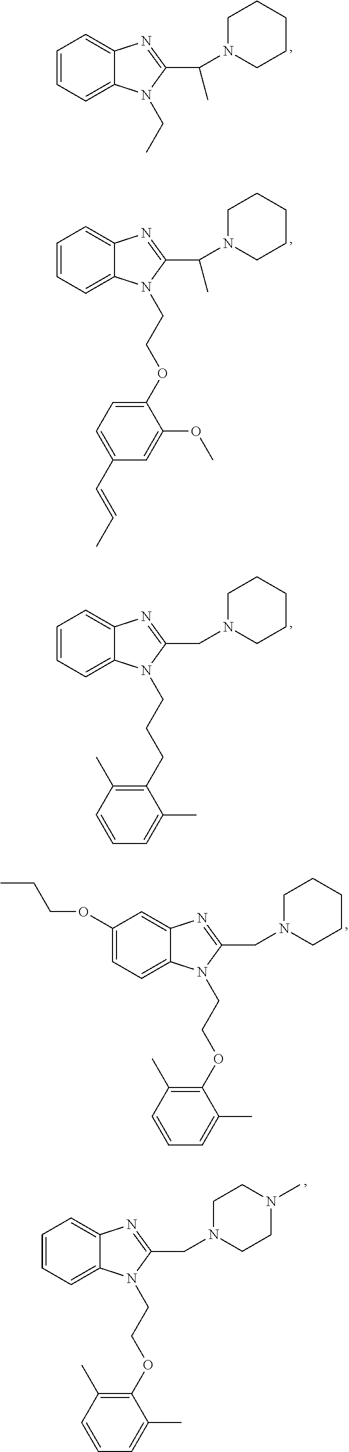 [SMILES: CCn1c(C(C)N2CCCCC2)nc2ccccc21 ; C/C=C/c1ccc(OCCn2c(C(C)N3CCCCC3)nc3ccccc32)c(OC)c1 ; Cc1cccc(C)c1CCCn1c(CN2CCCCC2)nc2ccccc21 ; CCCOc1ccc2c(c1)nc(CN1CCCCC1)n2CCOc1c(C)cccc1C ; Cc1cccc(C)c1OCCn1c(CN2CCN(C)CC2)nc2ccccc21] 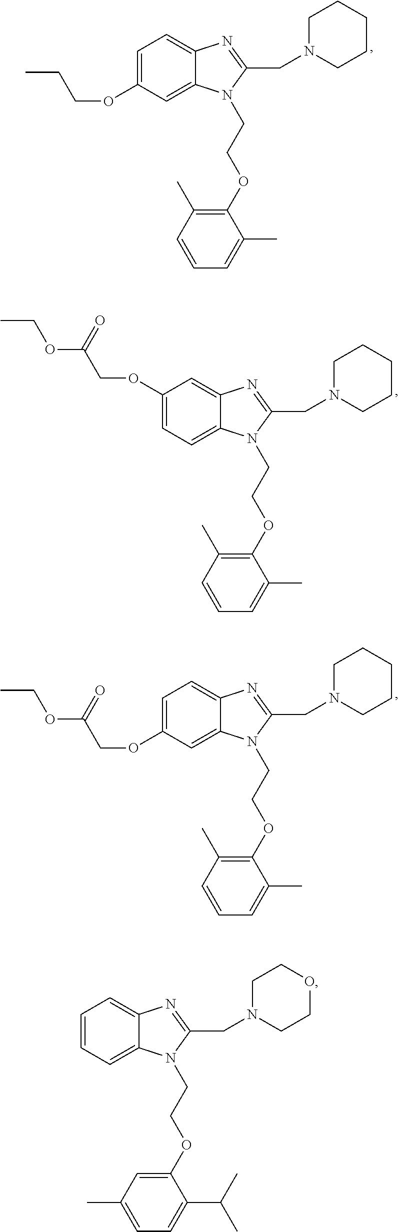 [SMILES: CCCOc1ccc2nc(CN3CCCCC3)n(CCOc3c(C)cccc3C)c2c1 ; CCOC(=O)COc1ccc2c(c1)nc(CN1CCCCC1)n2CCOc1c(C)cccc1C ; CCOC(=O)COc1ccc2nc(CN3CCCCC3)n(CCOc3c(C)cccc3C)c2c1 ; Cc1ccc(C(C)C)c(OCCn2c(CN3CCOCC3)nc3ccccc32)c1] 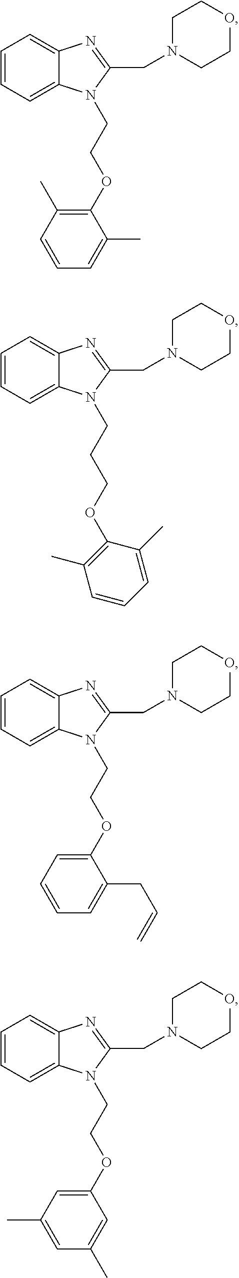 [SMILES: Cc1cccc(C)c1OCCn1c(CN2CCOCC2)nc2ccccc21 ; Cc1cccc(C)c1OCCCn1c(CN2CCOCC2)nc2ccccc21 ; C=CCc1ccccc1OCCn1c(CN2CCOCC2)nc2ccccc21 ; Cc1cc(C)cc(OCCn2c(CN3CCOCC3)nc3ccccc32)c1] 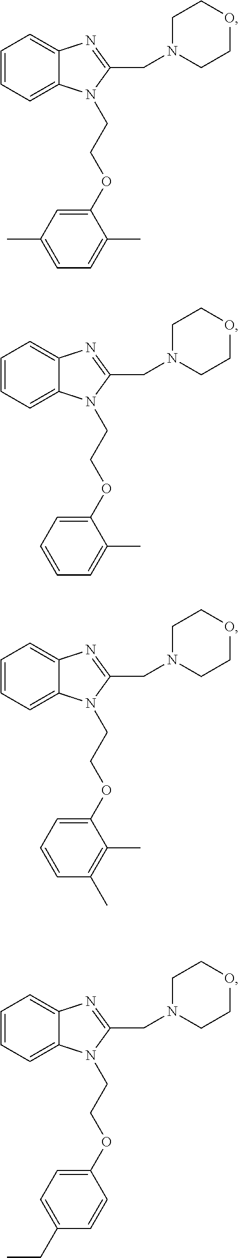 [SMILES: Cc1ccc(C)c(OCCn2c(CN3CCOCC3)nc3ccccc32)c1 ; Cc1ccccc1OCCn1c(CN2CCOCC2)nc2ccccc21 ; Cc1cccc(OCCn2c(CN3CCOCC3)nc3ccccc32)c1C ; CCc1ccc(OCCn2c(CN3CCOCC3)nc3ccccc32)cc1] 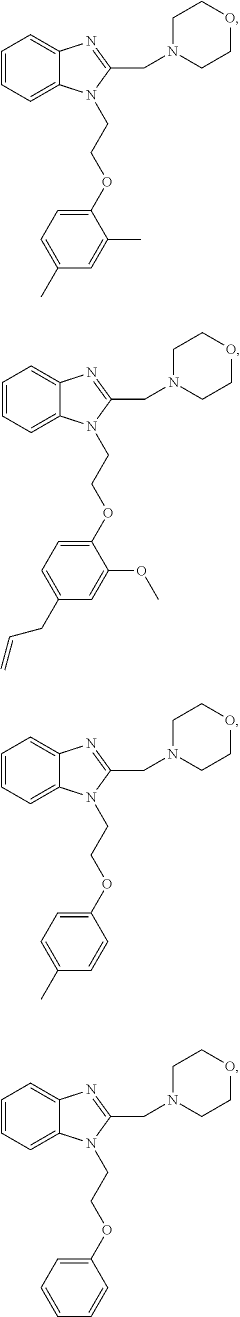 [SMILES: Cc1ccc(OCCn2c(CN3CCOCC3)nc3ccccc32)c(C)c1 ; C=CCc1ccc(OCCn2c(CN3CCOCC3)nc3ccccc32)c(OC)c1 ; Cc1ccc(OCCn2c(CN3CCOCC3)nc3ccccc32)cc1 ; c1ccc(OCCn2c(CN3CCOCC3)nc3ccccc32)cc1] 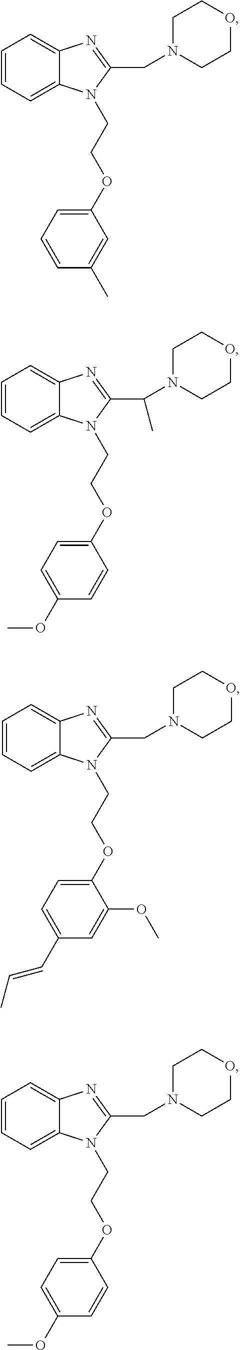 [SMILES: Cc1cccc(OCCn2c(CN3CCOCC3)nc3ccccc32)c1 ; COc1ccc(OCCn2c(C(C)N3CCOCC3)nc3ccccc32)cc1 ; C/C=C/c1ccc(OCCn2c(CN3CCOCC3)nc3ccccc32)c(OC)c1 ; COc1ccc(OCCn2c(CN3CCOCC3)nc3ccccc32)cc1] 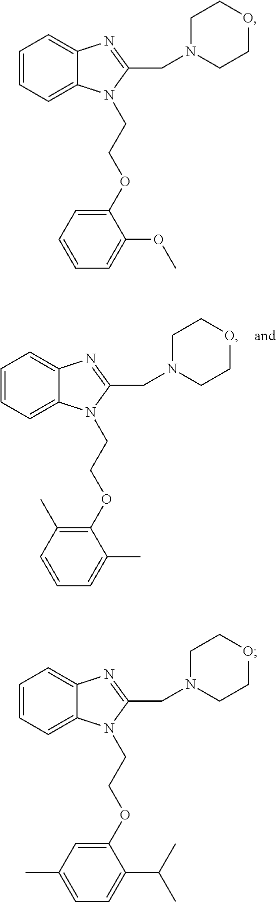 [SMILES: COc1ccccc1OCCn1c(CN2CCOCC2)nc2ccccc21 ; Cc1cccc(C)c1OCCn1c(CN2CCOCC2)nc2ccccc21 ; Cc1ccc(C(C)C)c(OCCn2c(CN3CCOCC3)nc3ccccc32)c1]
or a pharmaceutically acceptable salt thereof.
